(19) United States
(12) Patent Application Publication
(10) Pub. No.: US 2012/0150030 A1
(43) Pub. Date: Jun. 14, 2012

(54) Instruments, Methods and Systems for Harvesting and Implanting Cartilage Material

(75) Inventors: John S. Reach, JR., Guilford, CT (US); Ronald Litke, Sandy Hook, CT (US); Stephen Santangelo, Wallingford, CT (US); Stephen Maguire, Shelton, CT (US)

(73) Assignees: ACCELERATED ORTHOPEDIC TECHNOLOGIES, INC., Guilford, CT (US); YALE UNIVERSITY, New Haven, CT (US)

(21) Appl. No.: 13/383,990

(22) PCT Filed: Jul. 15, 2010
(86) PCT No.: PCT/US2010/042154

§ 371 Date: Mar. 2, 2012

Related U.S. Application Data
(60) Provisional application No. 61/225,833, filed on Jul. 15, 2009.

(51) Int. Cl. A61B 6/00; A61B 17/32

(52) U.S. Cl. 600/427; 606/170

(57) ABSTRACT

The present disclosure provides instruments and systems for accessing and removing hyaline cartilage from desired donor sites. The present disclosure also provides instruments/systems for implantation of hyaline cartilage grafts, e.g., to fill osteochondral defects. The apparatus/systems may be used in connection with mapping techniques and systems. Thus, in exemplary embodiments of the present disclosure, a clinician may be guided in his use of the disclosed apparatus/systems by articular joint surface mapping data in locating/identifying harvest sites for “best fit” grafts, i.e., grafts that exhibit desired geometric and/or surface attributes for use in particular implantation site(s). Alternatively, the disclosed instruments/systems may be employed to access anatomical sites independent of such mapping techniques/systems.

### BACKGROUND

1. Technical Field

The present disclosure is directed to instruments, methods and systems for use in harvesting cartilage from donor sites. More particularly, the present disclosure provides apparatus and systems that may be used by clinicians to acquire osteochondral grafts of desired shapes, sizes and/or depths in an efficient and reliable manner, and to implant such grafts in desired locations.

2. Background Art

Articular cartilage is a complex structure that, once damaged, has little capacity for permanent repair. One technique that has received attention for addressing cartilage-related issues involves repair with living hyaline cartilage through osteochondral autograft transplant. The procedure is known as mosaicplasty and generally involves removing injured tissue from a damaged area. One or more cylindrical sockets are drilled into the underlying bone and a cylindrical plug graft—consisting of healthy cartilage from the knee—is implanted in each socket.

As discussed in a commonly assigned PCT application entitled “Systems, Devices and Methods for Cartilage and Bone Grafting,” which published as WO 2009/154691 A9 (corrected version), commercially available instruments for use in mosaicplasty procedures are Acufex instruments available from Smith & Nephew, Inc. (Andover, Mass.), the COR System available from Innovasive Technologies (Marlborough, Mass.), and the Arthrex Osteochondral Autograft Transfer System available from Arthrex (Naples, Fla.). The content of the foregoing PCT application is incorporated herein by reference.

Despite efforts to date, a need remains for instruments and systems for efficient, effective and reliable access to desired cartilage sites and removal of desired cartilage tissue. In addition, a need remains for instruments/systems that facilitate cartilage access and/or removal in a minimally invasive manner. Still further, a need remains for instruments/systems that facilitate effective, efficient and reliable implantation of cartilage tissue, e.g., to fill osteochondral defects. These and other needs are met by the instruments/systems and associated methods disclosed herein.

### SUMMARY

The present disclosure provides instruments and systems for accessing and removing hyaline cartilage from desired donor sites. The present disclosure also provides instruments/systems for implantation of hyaline cartilage grafts, e.g., to fill osteochondral defects. The disclosed apparatus/systems may be used in connection with mapping techniques and systems of the type set forth in the commonly assigned PCT application entitled “Systems, Devices and Methods for Cartilage and Bone Grafting” (WO 2009/154691 A9; corrected version).

Thus, in exemplary embodiments of the present disclosure, a clinician may be guided in his use of the disclosed apparatus/systems by articular joint surface mapping data in locating/identifying harvest sites for “best fit” grafts, i.e., grafts that exhibit desired geometric and/or surface attributes for use in particular implantation site(s). Alternatively, the disclosed instruments/systems may be employed to access anatomical sites independent of such mapping techniques/systems. For purposes of the present disclosure, reference is made to the commonly assigned PCT application entitled “Systems, Devices and Methods for Cartilage and Bone Grafting” (WO 2009/154691 A9; corrected version) for purposes of advantageous data mapping systems and techniques that may be employed with the disclosed instruments/systems and associated methods.

In exemplary embodiments of the disclosed instruments/systems, one or more of the following features/functionalities are provided:
means for establishing referential orientation of instrumentation relative to anatomical location/defect, e.g., locking cannula assembly;
means for controlling geometry and/or depth of material removal at anatomical location; e.g., defect template(s) associated with cannula housing; bushing mechanism for controlling depth of cutting implement travel;
means for capturing information concerning surface contour of anatomical location, e.g., a surface contour tool featuring a plurality of circumferentially spaced, axially translatable rod/pin members and a centrally located plunger member for positioning within a defect, the surface contour tool adapted to key to a cannula assembly, or a balloon member surrounding a defect insert that is adapted to receive a curing agent;
means for excising a plug from a defect plug material, such plug exhibiting a geometry that substantially conforms to the surface topography surrounding the defect site and that substantially conforms to the geometry of the defect itself, e.g., a cutting tool associated with a surface contour tool that is adapted to key to a cannula assembly; and
means for implanting an excised plug in a defect.

In further exemplary embodiments of the disclosed instruments/systems, one or more of the following features/functionalities are provided:
means for accessing a defect region-of-interest at an angle relative to an elongated shaft (e.g., 90°), wherein a probe tip is associated with a pin that moves within a control member (e.g., defect template) associated with a handle member;
means for effectuating cutting functionality at an angle relative to an elongated shaft (e.g., 90°), wherein the cutting blade is adapted for movement relative to a distally-located housing between a recessed/shielded orientation and an operative orientation;
means for driving the cutting blade at an angle relative to an elongated shaft (e.g., 90°), e.g., a bevel gear drive mechanism, a rotating vane mechanism, and/or a belt/pulley mechanism.

In still further exemplary embodiments of the disclosed instruments/systems, one or more of the following features/functionalities are provided:
means for pointing to a defect location; and
means for effectuating cutting functionality at the desired defect location, wherein the foregoing functionalities are achieved utilizing in part a “four-bar” linkage mechanism.

Additional features, functions and advantages associated with the disclosed instruments, systems and methods will be apparent from the detailed description which follows, particularly when read in conjunction with the appended figures.

### BRIEF DESCRIPTION OF THE FIGURES

To assist those of skill in the art in making and using the disclosed instruments and systems, reference is made to the accompanying figures, wherein:

FIG. 1 is a schematic side view of an exemplary locking cannula assembly for use according to an illustrative embodiment of the present disclosure;

FIG. 1a is a schematic view of the distal end of the locking cannula of FIG. 1 illustrating interaction with a target anatomical location;

FIG. 2 is a schematic view of the locking cannula assembly of FIG. 1 with trocar removed;

FIG. 2A is a top view of the cannula housing of the locking cannula assembly of FIGS. 1 and 2, with an exemplary defect template positioned therein;

FIG. 3 is a schematic view of the locking cannula assembly of FIG. 2 with a cutter tool inserted therein;

FIG. 3A is side view of an exemplary interaction of a cutting tool with a cannula housing according to the present disclosure;

FIG. 3B is a top view of the exemplary cutting tool of FIGS. 3 and 3A forming a desired cut in an anatomical structure based on the defect template associated with the cannula housing;

FIGS. 4, 4A, 4B, 4C and 4d are schematic views of an exemplary surface contour tool that may be used in cooperation with the locking cannula assembly of the preceding figures;

FIGS. 5, 5A and 5B schematically depict exemplary cutting tool functionality for excising a plug from a donor graft material;

FIGS. 6, 6A and 6B depict exemplary instrumentation/methodology for delivery of an excised plug to a defect location;

FIGS. 7 and 7A-7E depict exemplary instrumentation for accessing a defect region at an angle relative to an elongated shaft;

FIG. 8 schematically depicts an exemplary bevel gear drive mechanism for use with the exemplary cutting assembly depicted in the preceding figures;

FIGS. 9 and 9A-9C depict an alternative exemplary cutting instrument for use in cutting at an angle relative to an elongated shaft;

FIGS. 10 and 10A-10D depict a further alternative exemplary cutting instrument for use in cutting at an angle relative to an elongated shaft;

FIGS. 11 and 11A depict an exemplary assembly for use in capturing topographical information form a surface;

FIGS. 12-14 depict an exemplary system for guidance of cutting actions relative to an anatomical location;

FIG. 15 depicts an exemplary template assembly for use according to a further exemplary embodiment of the present disclosure;

FIG. 16 depicts an alternative or complementary template assembly implementation;

FIG. 17 depicts a template member according to an exemplary embodiment of the present disclosure;

FIG. 18 depicts an exemplary graft harvesting device according to the present disclosure;

FIGS. 19-22 are views of the distal end of the exemplary graft harvesting device of FIG. 18;

FIGS. 23A and 23B are views of the proximal end of the exemplary graft harvesting device of FIG. 18;

FIG. 24 depicts a further exemplary fixture clamp according to the present disclosure;

FIGS. 25 and 26 depict the fixture clamp of FIG. 24 in conjunction with a pointer;

FIGS. 27 and 28 depict the fixture clamp of FIG. 24 in conjunction with a template member according to the present disclosure;

FIGS. 29-31 depict the fixture clamp/template member of FIGS. 27 and 28 in conjunction with a cutter assembly according to the present disclosure;

FIG. 32 depicts the exemplary fixture clamp/template member of FIGS. 27 and 28 positioned with respect to a talus;

FIGS. 33-35 depict an exemplary pin guide and punch assembly according to the present disclosure; and

FIGS. 36-40 depict an exemplary system according to the present disclosure that includes, inter alia, a four bar linkage mechanism.

### DESCRIPTION OF EXEMPLARY EMBODIMENT(S)

Instruments and systems for accessing, removing and/or implanting hyaline cartilage are provided herein. The disclosed apparatus/systems may optionally be used in connection with mapping techniques and systems of the type set forth in the commonly assigned PCT application entitled “Systems, Devices and Methods for Cartilage and Bone Grafting” (WO 2009/154691 A9; corrected version). The disclosed apparatus/systems provide effective, efficient and reliable systems for use in mosaicplasty.

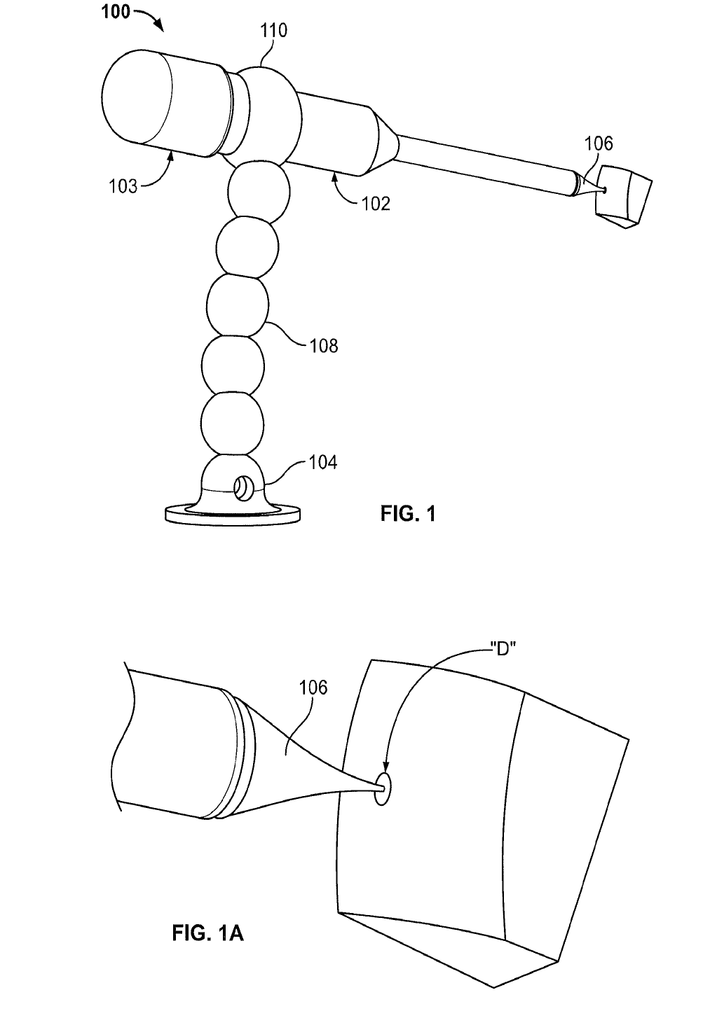

With initial reference to FIGS. 1 and 1A, an exemplary instrument 100 for use in accessing an osteochondral defect “D” is provided. Instrument 100 includes a cannula 102 that is adapted to be positioned relative to a target location, e.g., an osteochondral defect “D”. In this regard, instrument 100 includes a trocar 103 defining a trocar tip 106 that is adapted to extend from the distal end of locking cannula 102 for positioning within defect “D”. Instrument 100 is generally adapted to be rigidly mounted with respect to a fixed structure, e.g., a bed or the like. Thus, the base 104 of instrument 100 generally includes (or is adapted to cooperate with) a mounting mechanism for use in rigidly securing the base 104 relative to such fixed structure, e.g., a conventional clamping mechanism (not pictured).

Once the base 104 of instrument 100 is secured relative to an underlying structure, the trocar tip 106 is generally brought into position relative to defect “D”. Thus, instrument 100 generally supports/permits repositioning of trocar tip 106 relative to the fixed base 104. In the exemplary embodiment of FIGS. 1 and 1A, instrument 100 include a flexible shaft 108 that extends from base 104 to a mounting mechanism, e.g., a securement ring 110, that is adapted to engage/retain cannula 102. Once trocar tip 106 is positioned in a desired manner relative to defect “D”, flexible shaft 108 is locked in position, thereby fixing reference distances/orientations of cannula 102 relative to defect “D”.

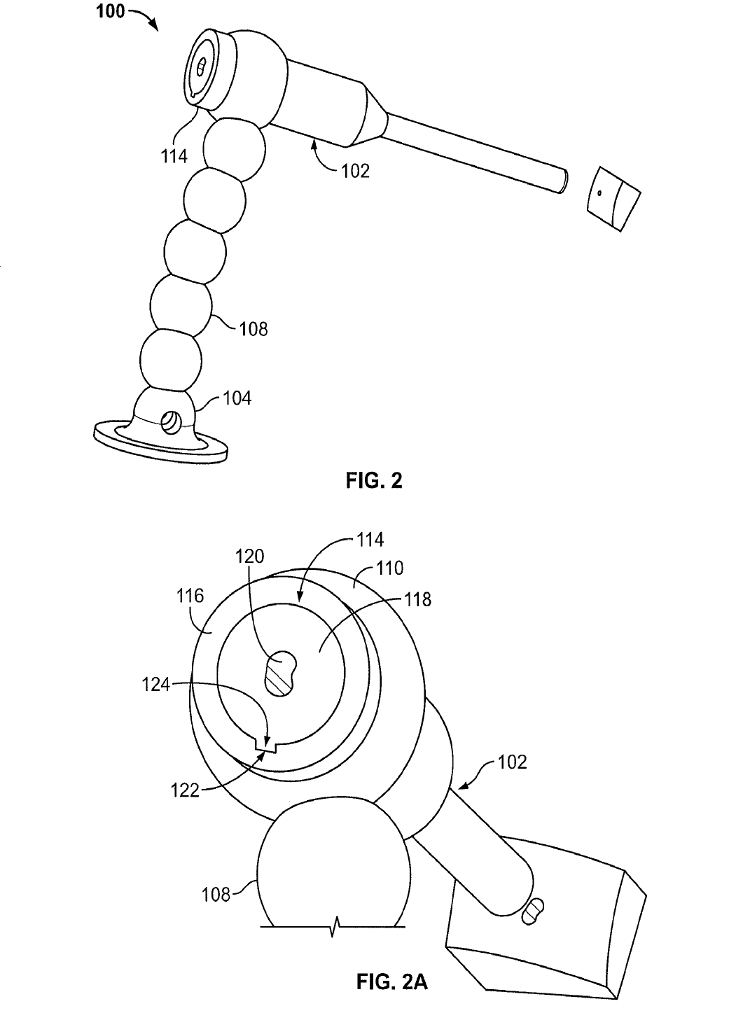

As shown in FIGS. 2 and 2A, the trocar 103 may be removed from cannula 102, thereby exposing the internal region defined by cannula housing 114. The proximal end of cannula housing 114 defines a keyed ring 116 that is adapted to receive a plurality of defect templates 118. Each defect template 118 advantageously defines an opening 120 that corresponds to a desired tissue removal geometry. Thus, for example, the geometry of opening 120 in the exemplary embodiment of FIG. 2A approximates an elliptical/oval shape. In use (and as described in greater detail below), a cutting tool inserted through opening 120 will be confined in its X-Y movement by the perimeter of the template opening. Alternative defect template geometries may be provided for use with the disclosed instrument 100. For example, a series/set of predefined defect template geometries may be supplied with or otherwise for use with the disclosed instrument 100. Alternatively (or additionally), customized defect template geometries may be created in response to specific anatomical criteria associated with a particular clinical procedure. Regardless, the interchangeable functionality associated with the disclosed defect templates greatly enhances the flexibility and customization associated with exemplary embodiments of the present disclosure.

Each defect template 118 advantageously includes keying feature(s) that is/are adapted to engage with the corresponding feature(s) defined by keyed ring 116. In the exemplary embodiment of FIG. 2A, the cooperative key structures comprise a substantially rectangular slot 122 formed in the keyed ring 116 and a cooperating protrusion 124 extending from the periphery of defect template 118. Of course, alternative keying structures/features may be employed according to the present disclosure, as will be readily apparent to persons skilled in the art. The keying functionality advantageously serves to fix the orientation of the opening geometry of defect template 118 relative to cannula 102 and, therefore, relative to defect “D”.

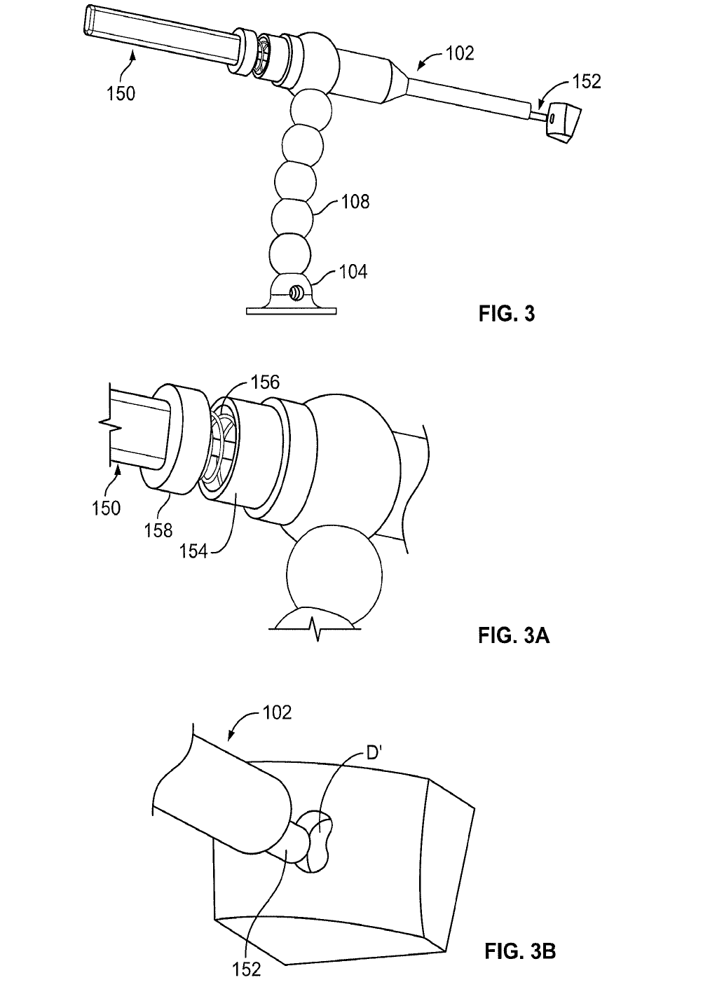

Turning to FIGS. 3, 3A and 3B, the disclosed cannula 102 is shown in association with an exemplary cutting tool 150 that includes a cutting element 152 at a distal end thereof. The shaft of cutting tool 150 passes through the opening 120 of defect template 118, which controls the X-Y movement of the cutting element 152 relative to the relevant anatomical surface. Thus, as shown in FIG. 3B, an enlarged defect region D′ is defined in the target anatomical region that substantially corresponds to the geometry of opening 120 in defect template 118. The enlarged defect region D′ may be characterized by alternative geometries through the selection/use of alternative defect templates 118, as discussed herein.

With reference to FIG. 3A, an exemplary mechanism for controlling the depth of travel for cutting element 152 is depicted. Thus, in the exemplary implementation depicted in FIG. 3A, cutting tool 150 includes cooperative bushing members 154, 158 with a spring member 156 captured therebetween. The relative axial travel permitted between bushing members 154, 158 defines the Z-axis travel of cutting element 152 relative to the target anatomical surface. In use, the cutting element 152 may advantageously bear against the anatomical surface (adjacent original defect “D”) when the spring member 156 in its rest/non-compressed state. In this orientation, bushings 154, 158 are at their greatest spacing. Thereafter, as the clinician advances the cutting tool 150 relative to cannula 102 (which is fixed in position and orientation), spring member 156 is compressed and bushing 158 moves axially toward bushing 154. When the two bushings 154, 158 are in abutting relation, the cutting tool 152 will have reached its maximum cutting depth relative to the anatomical surface.

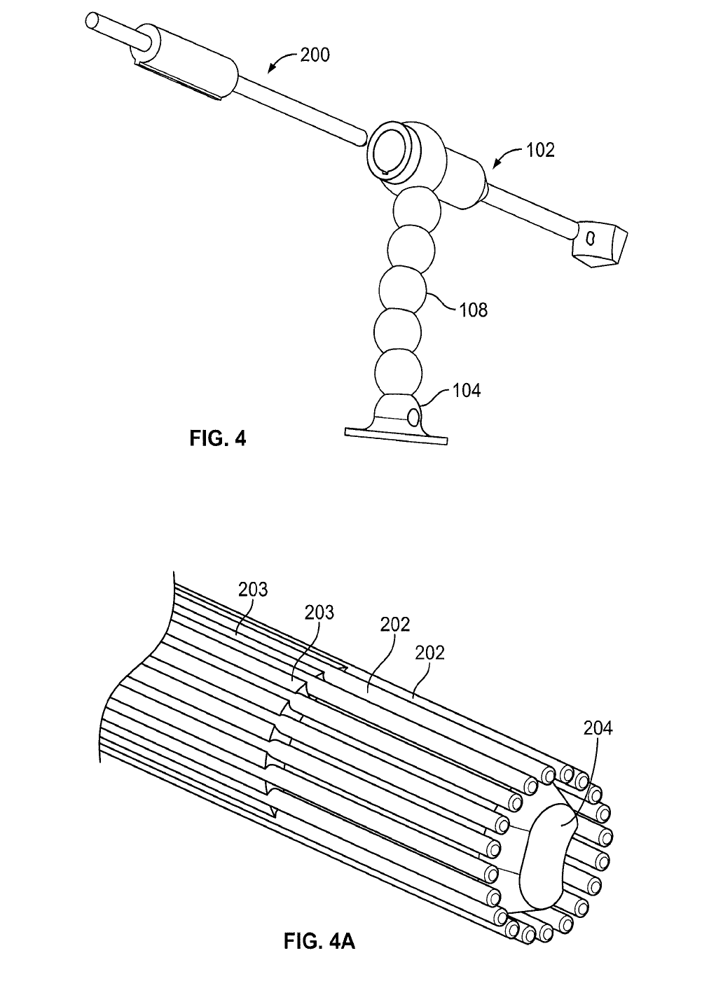

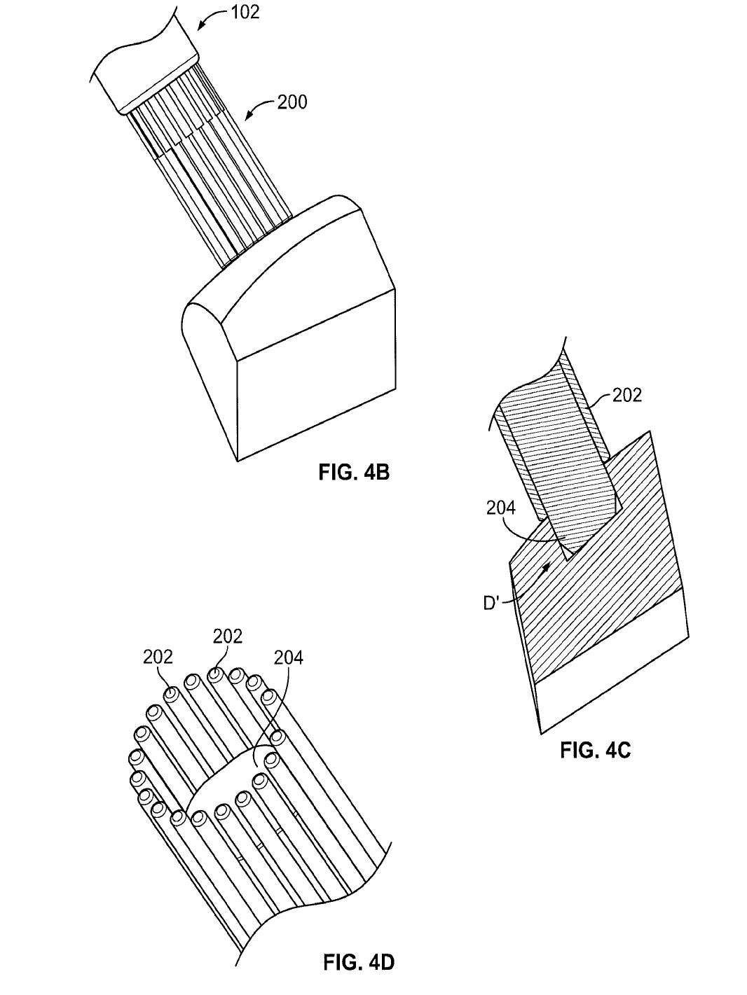

Turning to FIGS. 4, 4A, 4B, 4C and 4D, an exemplary surface contour tool 200 is schematically depicted. Surface contour tool 200 typically defines a substantially cylindrical structure for introduction through cannula 102 for engagement with an anatomical surface. For surface contour measurement purposes, surface contour tool 200 includes a plurality of circumferentially spaced, axially translatable rod or pin members 202 that are supported in channels defined by body structure 203 (see FIG. 4A). Surface contour tool 200 also generally includes a centrally located, axially translatable plunger member 204 that is positioned within body structure 203. In exemplary embodiments of the present disclosure, plunger member 204 defines a distal surface having a geometry that substantially corresponds to the geometry of the defect D′ such that plunger member 204 can be effectively positioned therewithin (see FIG. 4C). Of note, surface contour tool 200 generally includes “keying” functionality that is adapted to cooperate with the keying feature(s) associated with cannula housing 114, thereby permitting a clinician to ensure that the relative orientations of surface contour tool 200 and the anatomical region surrounding defect D′ are maintained, e.g., as surface contour tool 200 is introduced, removed and re-introduced to cannula 102.

At the opposite (proximal) end of surface contour tool 200, plunger member 204 may advantageously define a hollow cutting member (discussed below) of comparable geometry to the distal surface thereof, thereby permitting a plug member to be excised from a substrate having a geometry that substantially corresponds to the geometry of D′.

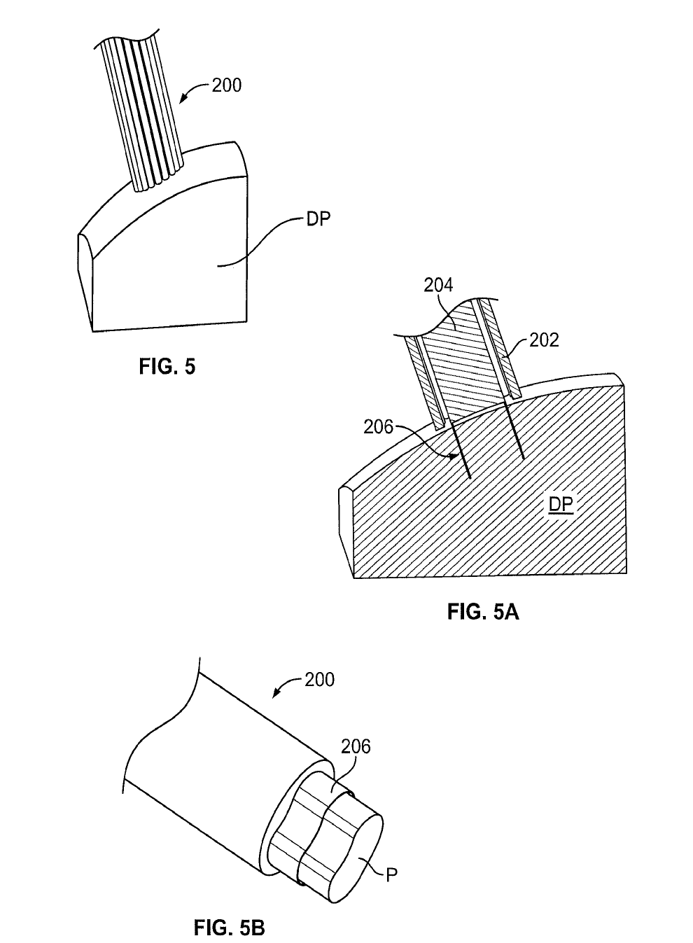

With further reference to FIGS. 4B-4D, rod members 202 are adapted to be brought into engagement with an anatomical surface and to thereby capture the geometric contour/topography thereof. Each rod member 202 translates independently of the remaining rod members 202, thereby permitting the plurality of rod members 202 to accurately reflect/capture the surface geometry/contour of a surface with respect to the substantially circle of contact/engagement. Once locked in place through a locking mechanism (not pictured), the rod members 202 allow clinicians to translate such surface geometry/contour to other surfaces for matching purposes. At the proximal end of surface contour tool 200, the rod/pin members 202 define a “negative” image of the anatomical surface geometry/contour. The number of rod members 202 included in the design of surface contour tool 200 is typically selected to maximize the instrument's ability to capture surface geometry/contour without sacrificing requisite stiffness/rigidity of the individual rod members. For example, exemplary implementations of the disclosed surface contour tool 200 include 12-40 rod members, although the present disclosure is not limited by or to such exemplary implementation. Turning to FIGS. 5, 5A and 5B, an exemplary implementation of the present disclosure adapted to capture a plug “P” for introduction to defect D′ is provided. In particular, the disclosed implementation contemplates additional functionality associated with surface contour tool 200, whereby a cutting member 206 is associated with the opposite end of plunger member 204. Indeed, as most clearly shown in FIG. 5B, cutting member 206 advantageously features a geometry that substantially corresponds to the geometry of defect D′ (and, by extension, the distal surface of plunger member 204 discussed with reference to the preceding figures). In use, the clinician may reverse plunger member 204 relative to rod/pin members 202, such that the captured surface geometry/contour of an anatomical surface is reflected by such rod/pin members 202 surrounding the cutting member 206.

Thus, the rod/pin members 202 may be brought into engagement with the surface of donor plug material “DP” (see FIGS. 5 and 5A) and moved/reoriented until such time as the geometry/contour of the donor plug material roughly approximates/matches the captured geometry/contour of the anatomical region surrounding defect D′. The donor plug material/donor graft may be pre-excised from an appropriate anatomical location (e.g., using the mapping technology disclosed in the appended PCT application), and the plug excision steps described herein may be performed in a location independent from the patient/source of the donor graft. Once a desired location/region on the donor plug material DP is located, the cutting member 206 is advanced relative to plunger member 204 into the plug material so as to excise a plug “P” of the desired geometry for introduction to the defect D′ (see FIG. 5B).

Of note, the present disclosure contemplates a fully customizable system for creation of a defect D′ and excision of an appropriate plug P to substantially match the geometry of defect D′. Thus, according to exemplary embodiments of the present disclosure, a customized defect template 118 and surface capture tool 200 may be fabricated based on specific aspects of a clinical procedure and/or patient. The customized defect template 118 and surface capture tool 200 would be fabricated such that the plunger member 204 and the cutting member 206 substantially match the geometry of the opening 120 defined in the defect template 118. Conventional fabrication techniques would be employed to mold/forge/machine the desired components such that the geometries of the noted components substantially correspond.

Alternatively, the present disclosure contemplates system(s) for creation of a defect D′ and excision of an appropriate plug P to substantially match the geometry of defect D′ wherein predetermined geometries most commonly encountered in clinical applications are manufactured, stocked and supplied to clinicians. According to this alternative approach, a plurality of predefined defect template geometries may be selected to encompass various clinical needs, e.g., ellipses/ovals of varying lengths, widths and peripheral irregularities. Based on the predefined template geometries, corresponding surface contour tools may be fabricated so as to facilitate harvesting of appropriately dimensioned plugs.

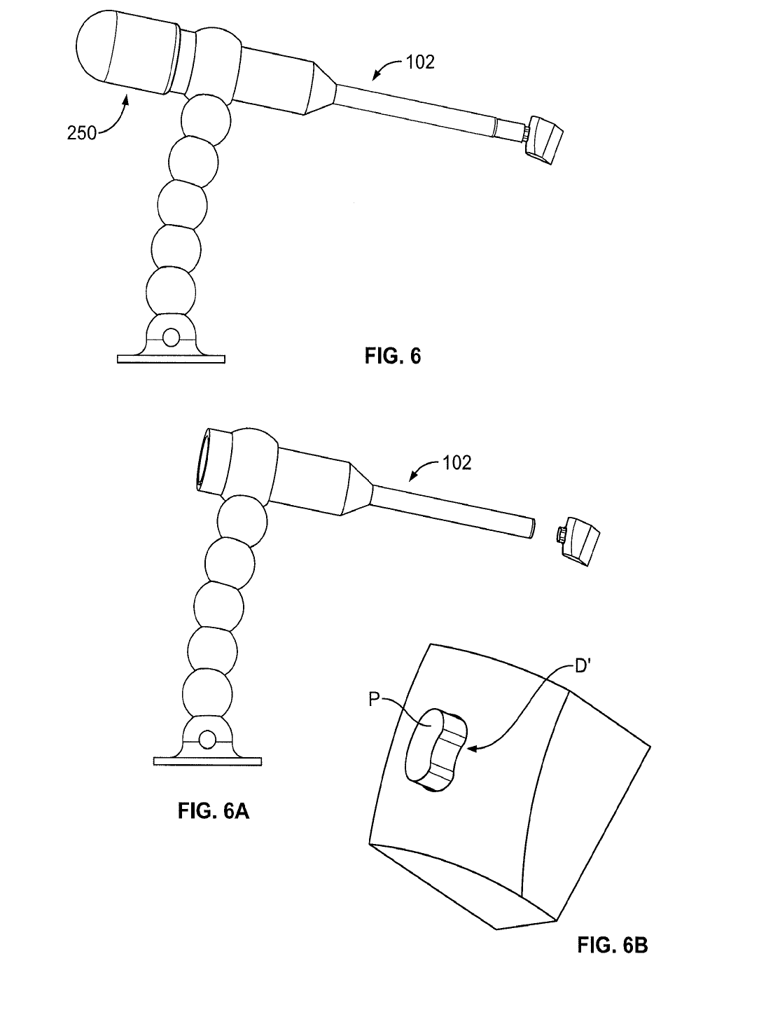

Once the plug P is obtained/excised, the clinician generally moves forward with implantation thereof in the defect D′. With reference to FIGS. 6, 6A and 6B, a plug insertion tool 250 may be used to introduce the plug P into the defect D′. The plug insertion tool 250 is adapted to key to the cannula housing 114 so as to ensure alignment of the plug P relative to the defect D′ (and the surrounding surface topography). The depth of plug insertion is generally controlled by interaction of the plug insertion tool 250 with the cannula housing 114. An axially movable plunger member (not pictured) is generally included in insertion tool 250 to facilitate advancement of plug P into defect D′. In exemplary implementations of the present disclosure, surface contour tool 200 may function as a plug insertion tool 250. After introduction of the plug P to the defect D′, a tamping tool (not pictured) may be used to fully seat the plug P within the defect D′. Once fully seated, the plug P exhibits a surface topography that closely approximates the topography of the anatomical surface surrounding the region of defect D′ (now filled with plug P), thereby greatly enhancing the efficacy of the disclosed mosaicplasty procedure.

In certain clinical procedures/environments, it may be desirable to obtain cartilage plug material from anatomical locations that are relatively difficult to access. Thus, for example, it may be desirable to access plug material in the talus bone (after distending the ankle relative to the talus bone). In such circumstances, it may be desirable to remove plug material at an angle relative to the plane of access, e.g., at an angle at or approaching 90° relative to the plane of access. According to the present disclosure, various exemplary instruments are provided for facilitating access to such locations and obtaining and/or implanting plug materials with respect to such locations.

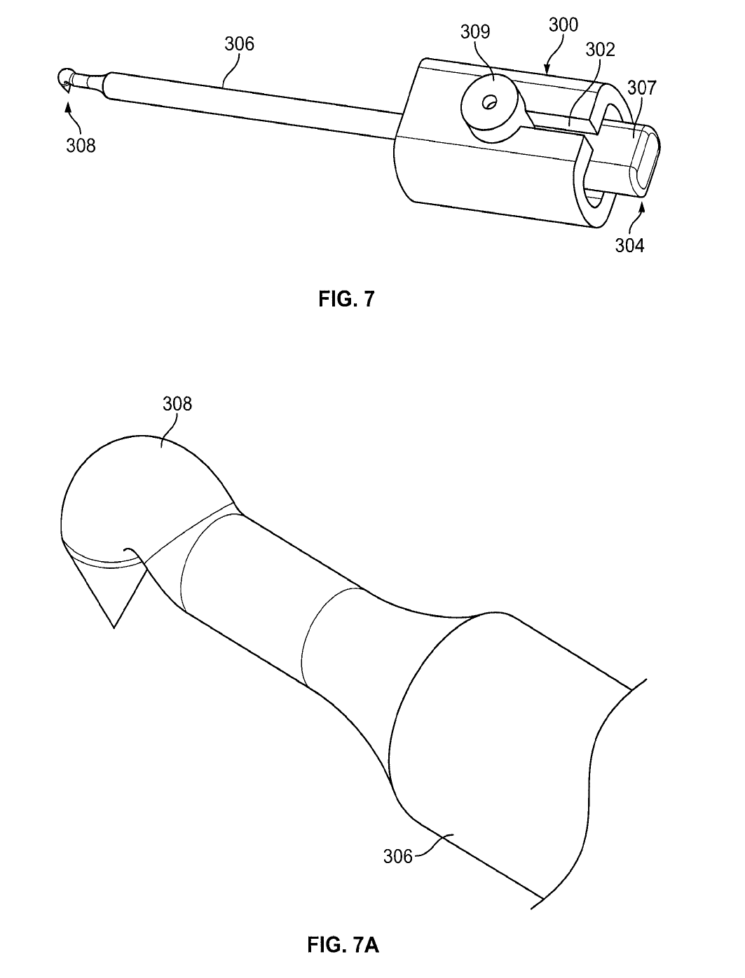

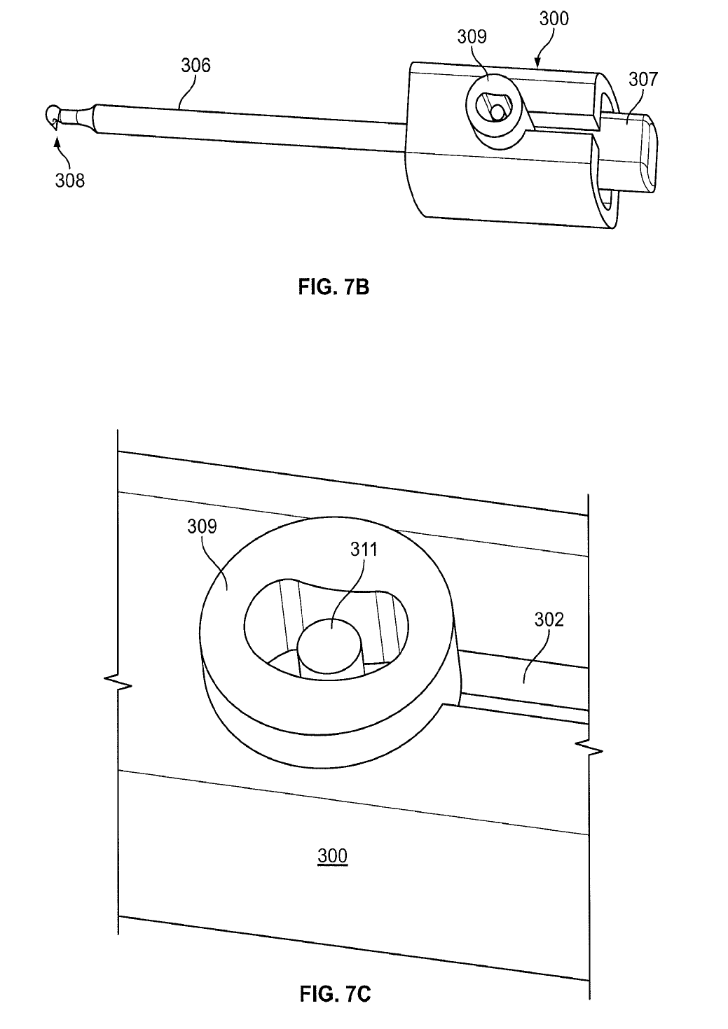

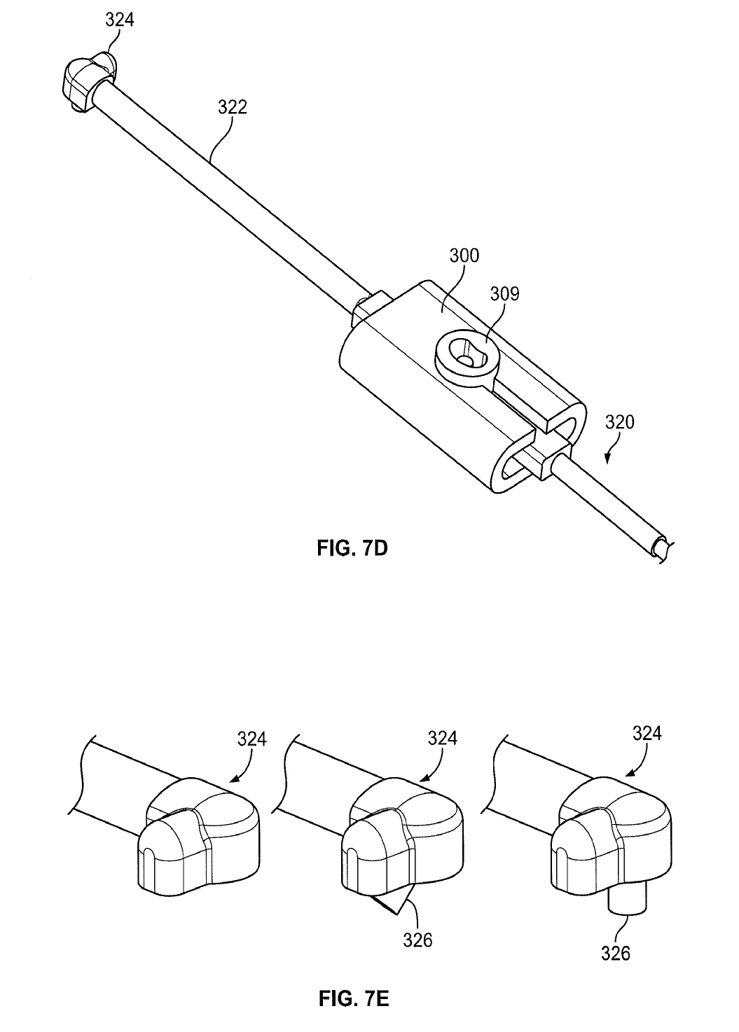

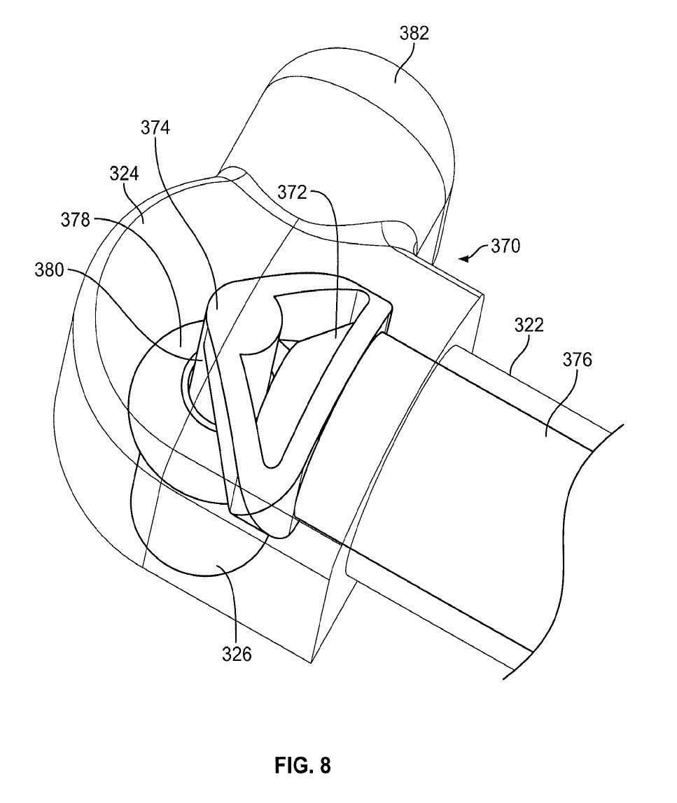

Thus, with reference to FIGS. 7, 7A, 7B, 7C, 7D and 7E, an exemplary system for accessing a defect site and positioning a cutting tool in proximity thereto for establishing an enlarged defect defining a predetermined geometric pattern. The disclosed system includes a handle 300 that defines an interior region for receipt of various tools, as described herein. Handle 300 is typically locked relative to a fixed structure, e.g., a distractor or other fixturing (not pictured), and maintains its position/orientation relative to the target anatomy throughout the disclosed procedure. In the exemplary embodiment disclosed herein, handle 300 defines a slot 302 that facilitates positioning/orientation of inserted tools.

With initial reference to FIGS. 7 and 7A, handle 300 initially receives a probe 304 that defines an elongated shaft 306, a substantially rectangular handle region 307, and a distally positioned probe tip 308 extending from shaft 306. Interchangeable control members 309 may be positioned on handle 300 (by sliding into position along slot 302) such that the control member 309 extends upwardly from handle region 307 and is accessible to a clinician above handle 300. Control member 309 defines an opening that is adapted to receive a pin 311 (see FIG. 7C) that is fixed relative to the shaft/probe tip, such that movement of pin 311 within the opening of control member 309 translates to corresponding movement of probe tip 308. By movement of pin 311 relative to control member 309, a clinician is able to explore the geometric contours of a defect region with probe tip 308. Of note, movement of the pin 311 relative to the opening of the control member 309 may relate to movement of the probe tip by a factor of about 1:3, although the present disclosure is not limited by or to such relationship.

In the exemplary embodiment of FIGS. 7 and 7A, probe tip 308 is oriented at a right angle relative to shaft 306. However, the present disclosure is not limited by or to such angular orientation. Rather, probe tip 308 may be oriented at various angles relative to shaft 306 without departing from the spirit or scope of the present disclosure.

In use, the shaft 306 is generally inserted to a desired anatomical location such that probe tip 308 is positioned adjacent/above a defect region-of-interest. The geometry of the defect region may be traced with probe tip 308 through movement of pin 311 within control member 309. According to exemplary embodiments of the present disclosure, an initial control member 309 with a non-specific opening geometry (e.g., an enlarged circle) may be employed to permit broad/unencumbered probing of a defect region. Thereafter, based on the clinicians observations with respect to the initial tracing of the probe tip relative to the defect region, a second control member 309 may be selected (or fabricated as a customized item) that defines an opening substantially corresponding to the overall geometric characteristics of the defect region (ensuring that the pre-existing defect region will be captured within the travel range permitted by the selected control member 309.

The selected control member 309 functions as a defect template for purposes of the disclosed system/methodology. For example, with reference to FIGS. 7B-7D, an exemplary control member 309 (defect template) is associated with handle 300, such control member 309 defining an opening that approximates an elliptical geometry with arcuate opposed faces. Pin 311—which is positioned within such elliptical opening—is free to trace such geometry. In use, after selecting the noted control member 309, the clinician may translate probe tip 308 relative to the defect to ensure that an appropriate “defect template” has been selected. If not, further selections may be undertaken until an appropriate geometry is in place. Of note, in exemplary embodiments of the present disclosure, implementation of various control members 309 is undertaken by sliding the control member proximally relative to housing 300 (with pin 311 captured therewith), thereby withdrawing the elongated shaft 306 from the anatomical region, disassociating the pin 311 from the opening in the control member 309, associating pin 311 with an opening associated with a second control member 309, and sliding the new control member 309 (with pin 311 captured therewithin) along slot 302 to the desired location on handle 300.

Once the clinician is satisfied with the selected control member 309, probe 304 is generally removed from handle 300 (which remains fixed relative to an underlying fixture) and cutter assembly is introduced to handle 300. Alternative cutting assemblies may be used according to the present disclosure.

In a first exemplary implementation and with reference to FIGS. 7D and 7E, a rotating cutter assembly 320 is provided that defines an elongated shaft 322, a cutter housing 324 and a cutting blade 326 at a distal end thereof. The rotating cutter assembly 320 is associated with a pin that is positioned within control member 309 (see FIG. 7D). Movement of the pin within the opening in control member 309 controls travel of the cutting blade 326 relative to the anatomical region-of-interest. In use, the cutting blade 326 is adapted to be rotated into a cutting position, i.e., at an orientation of 90° relative to shaft 322, from a recessed position within cutter housing 324. Of note, the exemplary cutter housing 324 defines a 90° jog at the distal end of shaft 322 to accommodate rotation of the cutting blade 326 into a fully recessed/protected orientation. Rotation of the cutting blade 326 into an operative orientation (see right-most schematic depiction in FIG. 7E) is effectuated through manipulation of the proximal region of rotating cutter assembly 320. Once rotated to the operative position, the cutting blade 326 is automatically positioned in a proper orientation relative to the defect-of-interest based on the relative orientation established by handle 300.

With reference to FIG. 8, an exemplary mechanism for controlling the orientation of cutting blade 326 and for delivering drive force thereto is schematically depicted. More particularly, cutter housing 324 is fixedly mounted with respect to cylindrical sleeve 322. The exemplary assembly includes a cylinder 376 rotatably positioned within cylindrical sleeve 322 and fixedly connected to frame 374. Frame 374 defines a hollow region within which drive shaft 372 can operate, as discussed below. Frame 374 supports rotating shaft 380 upon which cutting blade 326 is mounted. Rotation of cylinder 376 is controlled from the proximal end of cutting assembly 320. Based on 90° counter-clockwise rotation of cylinder 376 relative to cylindrical sleeve 322, cutting blade 326 will rotate from the deployed orientation shown in FIG. 8 to a recessed orientation within the jog portion 382 of cutter housing 324. By reversing such rotational motion of cylinder 376, cutting blade 326 may be brought back into the deployed orientation of FIG. 8. Detent mechanisms (or like locking structures) are typically provided at the proximal end of cutting assembly 320 to releasably secure the cutting blade 326 in one or the other orientation, as described herein.

With further reference to FIG. 8, with the cutting blade 326 in the deployed orientation, an exemplary control/drive mechanism 370 includes a drive shaft 372 that defines a bevel gear at a distal end thereof. A cooperative bevel gear 378 translates the motion of drive shaft 372 by 90°. In this way, cutting blade 326 may operate at an angular orientation relative to the elongated axis of the assembly. Based on movement of the pin within the control member (defect template) at the proximal end of the assembly, movement of the cutting blade 326 may be controlled to create an enlarged defect region having a desired geometry.

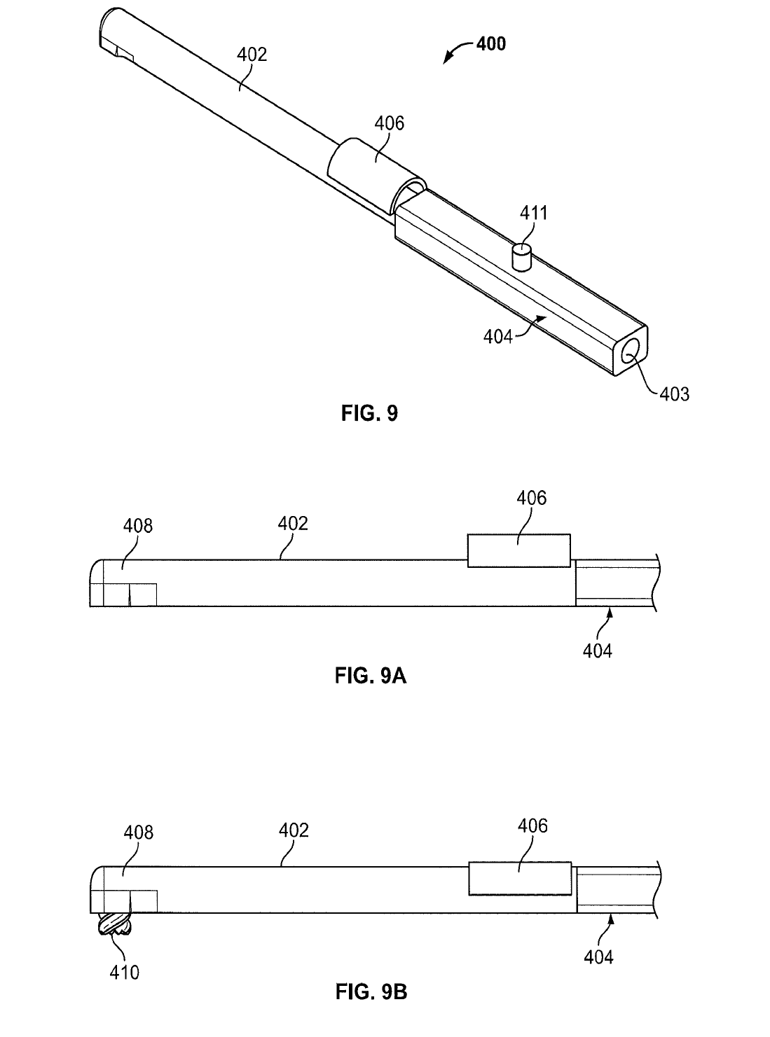

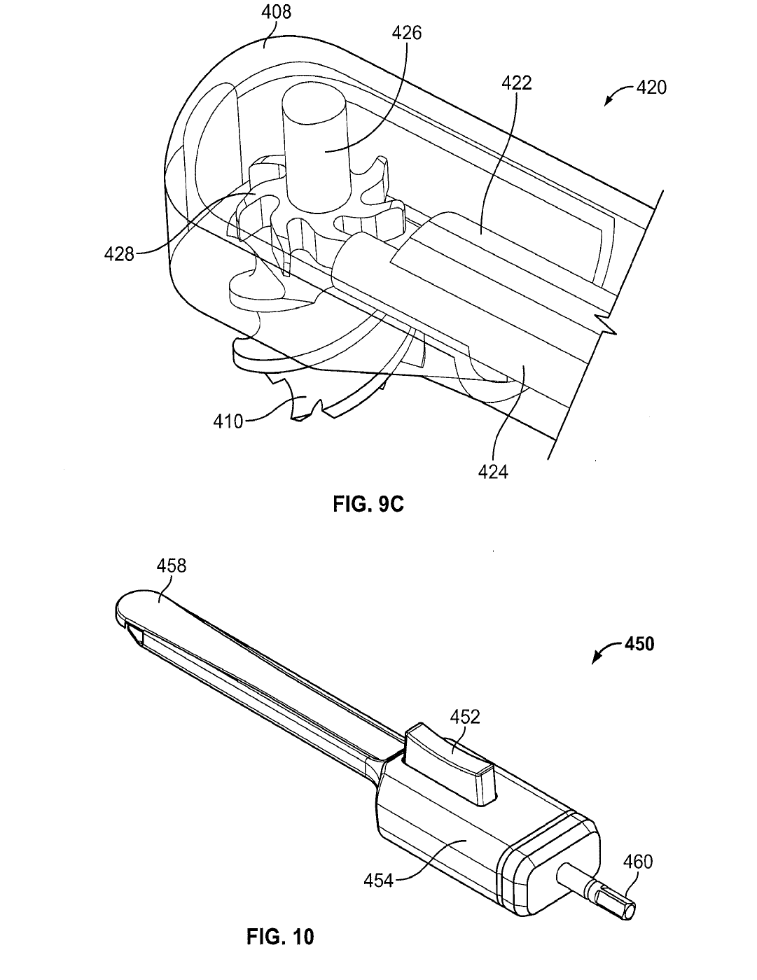

Turning to FIGS. 9 and 9A-9C, an alternative cutting assembly 400 is depicted. Cutting assembly 400 includes an elongated shaft 402 that cooperates with an inlet port 403 at a proximal end thereof. The handle region 404 also includes a pin 411 that is adapted to cooperate with a control member, as described hereinabove. A button 406 is positioned in association with (or adjacent to) the handle region 404. The button 406 cooperates with the cutting drive assembly positioned within elongated shaft 402 such that downward pressure on button 406 translates such cutting drive assembly downward relative to the elongated shaft 402, thereby deploying cutting blade 410 from a recessed orientation within cutter housing 408 (see FIG. 9A) to a deployed orientation extending from cutter housing 408 (see FIG. 9B).

An exemplary drive mechanism 420 for the cutting assembly 400 is schematically depicted in FIG. 9C. First, lever arm 422 cooperates with button 406 to move the cutting assembly downward into a deployed orientation. Return of the cutting assembly into a non-deployed orientation may be spring-biased (not pictured). Flow tube 424 is positioned below lever arm 422 and is in fluid communication with inlet port 403. The outlet of flow tube 424 is substantially aligned with rotating vane 428 which is mounted to a drive rod 426. Cutting blade 410 is also mounted with respect to drive rod 426. Thus, as high pressure fluid is introduced to inlet port 402 and flows through flow tube 424 into contact with rotating vane 428, rotation of drive rod 426 and cutting blade 410 are necessarily effectuated. The discharged fluid, e.g., water, enters the body in the region of the defect (together with other arthroscopic fluid that is already present). Travel of the cutting blade 410 relative to the anatomy is controlled by travel of pin 411 within the associated control member. Thus, the disclosed assembly is effective to achieve cutting functionality at an angle relative to the elongated shaft.

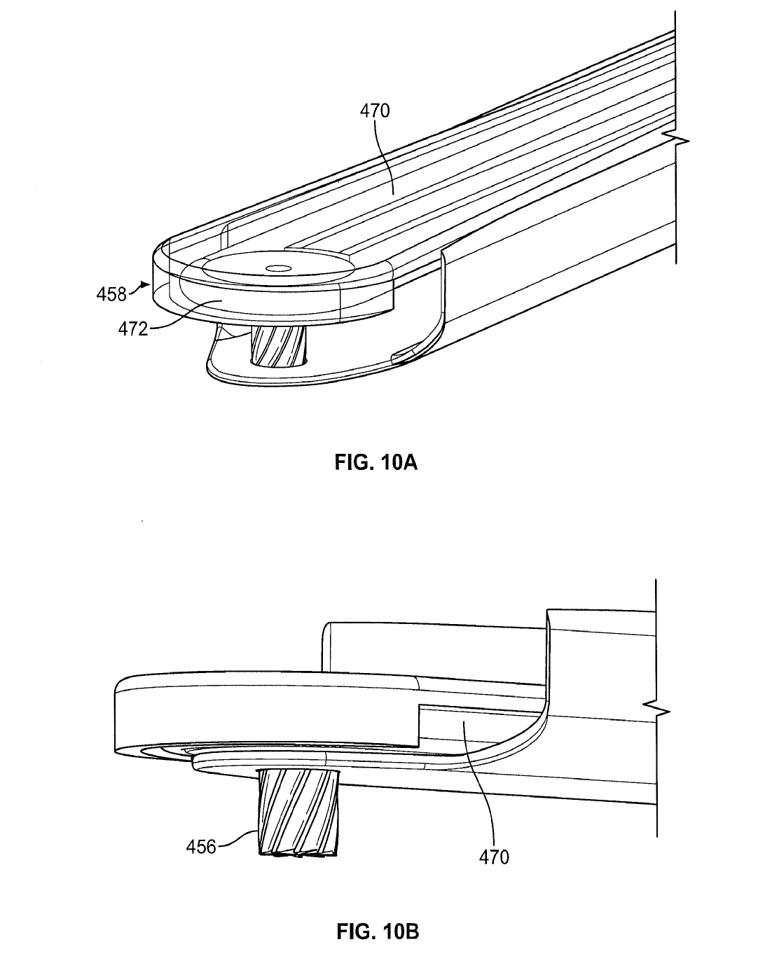

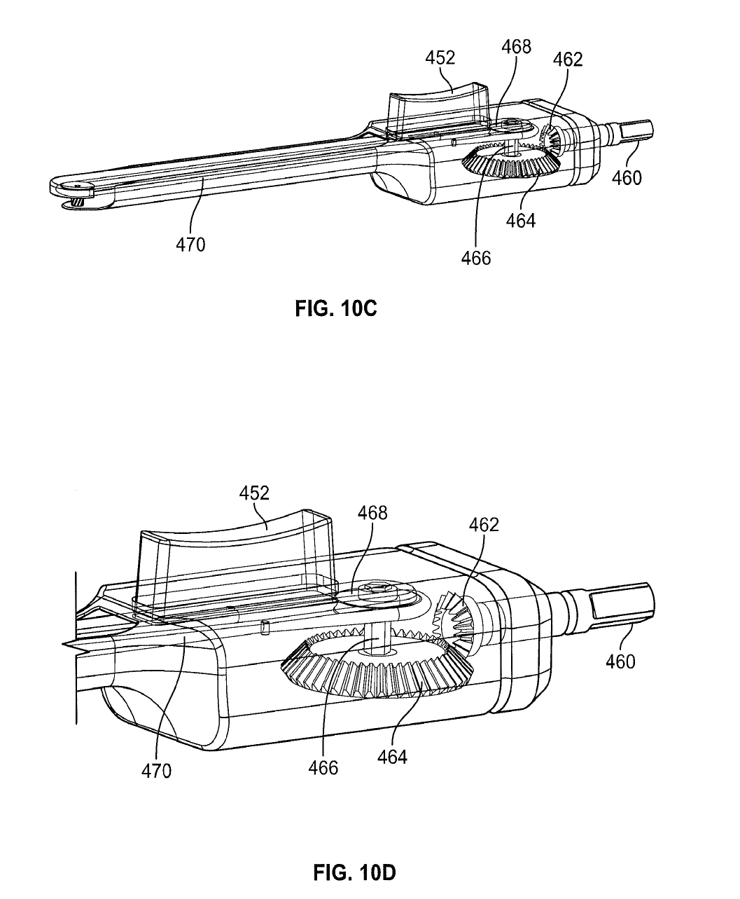

A further exemplary cutting assembly 450 is schematically depicted in FIGS. 10 and 10A-10D. Cutting assembly is driven by a belt and pulley system. Depression of button 452 relative to housing 454 deploys cutter 456 relative to cutter housing 458 (compare FIGS. 10A and 10B). A drive shaft 460 extends from the proximal end of housing 454 and through bevel gears 462, 464 translates rotational motion of drive shaft 460 to rotation of rod 466 internal to housing 454. Rod 466 cooperates with a first pulley wheel 468 that, through action of pulley belt 470, translates such rotational motion to second pulley wheel 472 positioned at the distal end of assembly 450. Rotational motion of second pulley wheel 472 is translated to cutting blade 456 which is mounted relative thereto. Thus, exemplary cutting assembly 450 provides a further exemplary instrument for effectuating cutting functionality at an angle relative to an elongated shaft.

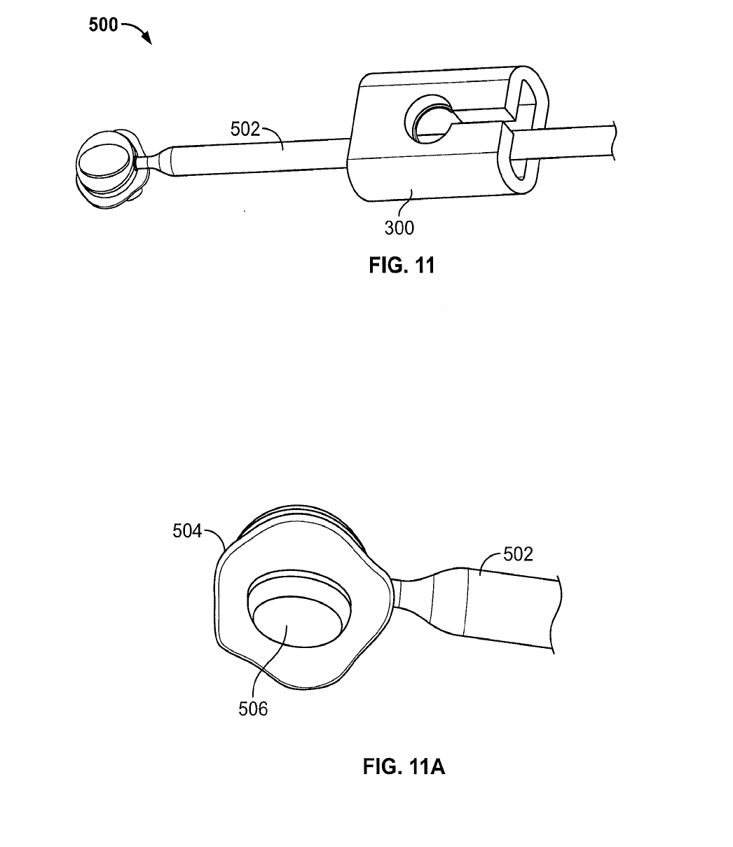

According to a further aspect of the present disclosure, an alternative apparatus for capturing the surface topography of a region adjacent a defect is provided. Thus, with reference to FIGS. 11 and 11A, the device 500 is adapted for use with handle 300 (describe above) and includes an elongated shaft 502 that is in fluid communication with an inflatable balloon member 504. Balloon member 504 is secured relative to and substantially surrounds a defect insert 506 that is oriented at an angle (e.g., 90°) relative to the elongated shaft 504. In use, the defect insert 506 is positioned within a defect-of-interest and the balloon member 504 is injected with a curing agent. The balloon member 504 is brought into and maintained in confronting engagement with the surface adjacent the defect while the curing agent sets.

Thereafter, the surface attributes of the cured balloon member 504 will correspond to the topographical features of the relevant surface. The cured balloon member 504 may thus be used to identify graft regions that will correspond to the topography surrounding the defect-of-interest.

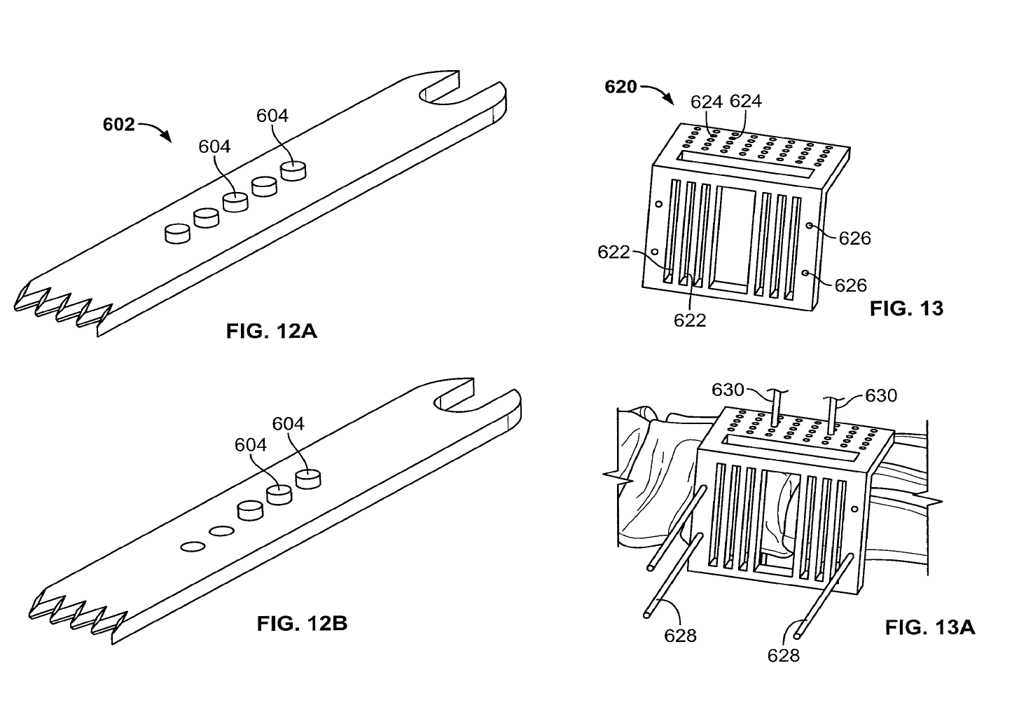

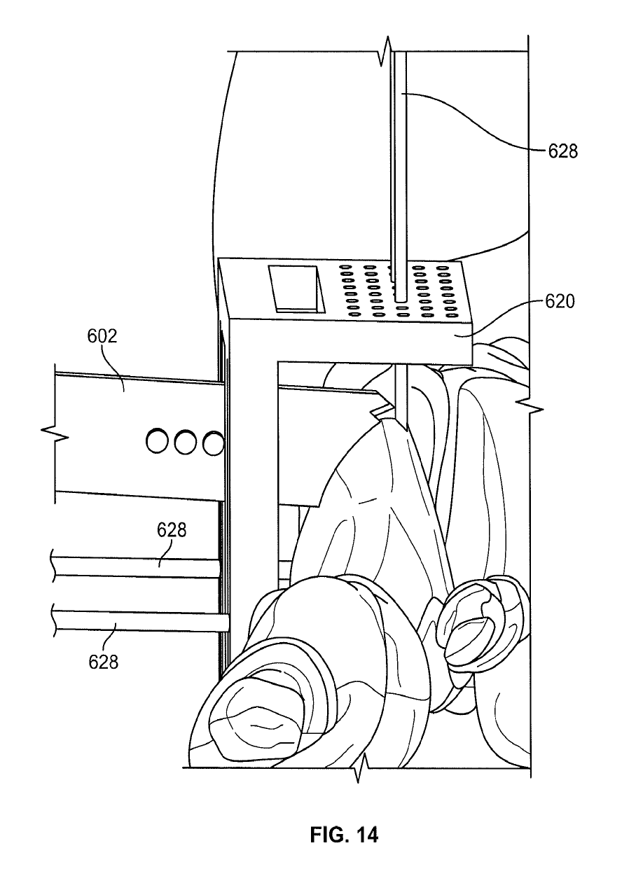

Turning to FIGS. 12-14, an exemplary system 600 for guiding cutting operations relative to anatomical region of interest are depicted. In particular, the disclosed system generally includes at least one guide blade 602 (see FIGS. 12A and 12B) that include knock-out plugs 604 for controlling the depth of cut in a clinical procedure. As shown in FIG. 14, cutting guide blade 602 is introduced to the bone to the degree permitted by the number of plugs 604 knocked out from guide blade 602. More particularly, guide blade 602 is introduced from the side of the anatomical region of interest until obstructed by a non-removed plug 604. Of note, the spacing of the plugs 604 on guide blade 602 corresponds to the K-wire holes associated with the cutting block (described below).

With reference to FIGS. 13 and 13A, cutting block 620 is substantially L-shaped and defines a series of vertically oriented slots 622 on a first side and a plurality of rows/columns of K-wire holes 624 on a second side thereof. The first side also includes mounting holes 626 along the edges thereof. In use, lateral/dorsal pins 628 are used to secure the cutting guide with respect to the anatomical region of interest and K-wires 620 are introduced through the holes formed in the first side of the cutting block 620 (see FIG. 13A). The guide blade 602 is introduced through a slot formed in the second side of the cutting guide 620 to the depth of the knocked out plugs 604. Thereafter, a blade (not pictured) can be inserted and a cut to the desired depth achieved.

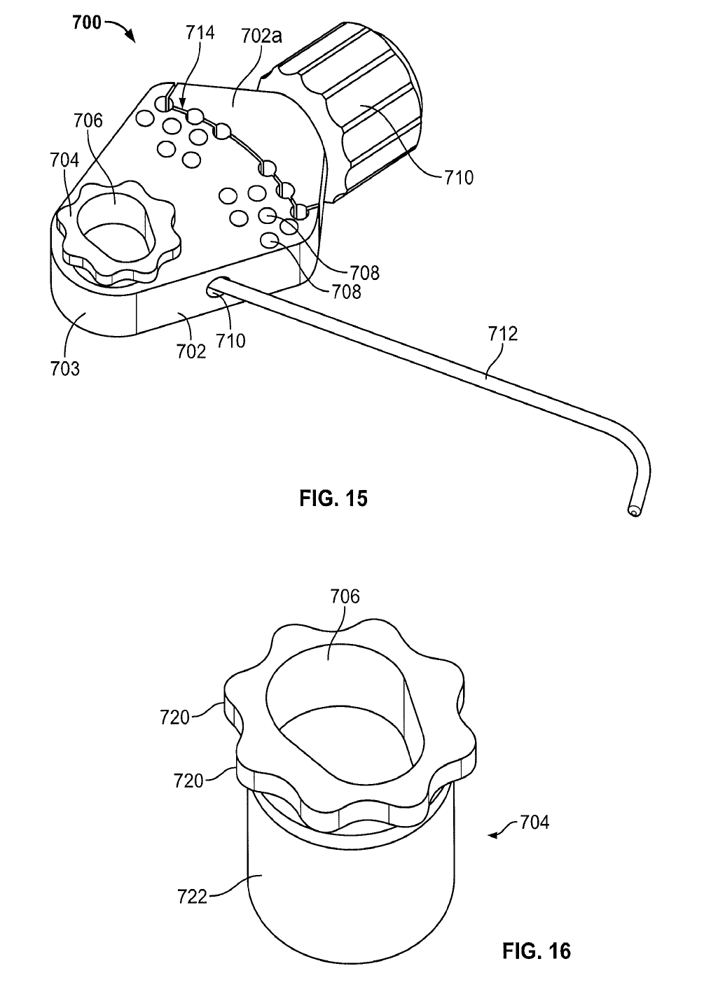

Turning to FIGS. 15-17, alternative template assemblies are provided according to the present disclosure. With initial reference to FIGS. 15-16, a template assembly 700 includes a substantially planar template body 702 that defines a template aperture 703 and a series of positioning apertures 708 that pass therethrough. Template aperture 703 is generally cylindrical in geometry, although alternative geometries may be employed. As shown in FIG. 15, template member 704 is positioned in template aperture 703 so as to associate unique template opening 706 with template assembly 700. Template member 704 is generally secured with respect to template body 702 by advancing a locking screw (not pictured) or like mechanism through locking aperture 710. A locking tool 712 may be used to advance the locking screw/mechanism relative to template member 704. In like manner, locking tool 712 may be used to release the locking screw/mechanism from engagement with template member 704 for removal and/or replacement of template member 704, e.g., with an alternative template member featuring a different template geometry.

As best seen in FIG. 16, template member 706 includes a plurality of outstanding ears 720 that facilitate manual interaction with template member, e.g., when positioning template member 706 relative to template body 702. Thus, in use, the clinician typically selects a template member 706 from a “library” of template members that feature different template geometries, such selection based on an effort to identify a template geometry that most closely approximates applicable clinical parameters. The template member 706 is introduced to template aperture 703 by introducing template cylinder 722 into template aperture 703. Radial positioning of template member 706 is undertaken by the clinician through manual rotation thereof (e.g., by positioning fingers between adjacent outstanding ears 720) so as to orient the template geometry of template member 704 in a desired position relative to the anatomy-at-issue. Template member 706 may be locked in position relative to template body 702 using locking tool 712, as described above.

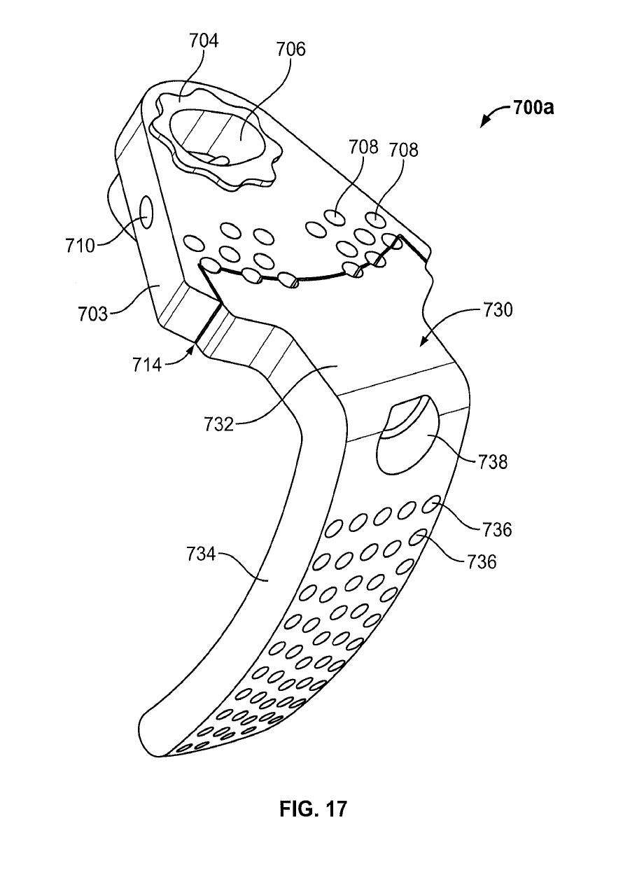

With further reference to FIG. 15, exemplary template body 702 includes template body extension 702a that is joined to template body 702 along interface 714. Template body 702 and template body extension 702a are joined relative to each other with a screw member 710 that includes a knurled knob. Of note, the template body 702 need not include an extension member, but may be fabricated as a single, unitary body. However, the implementation of FIG. 15 permits flexibility in design/use, as is apparent from the alternative template assembly 700a of FIG. 17.

More particularly, template assembly 700a includes template body 702 (as shown in FIG. 15), but with template body extension 702a removed. In place of template body extension 702a, template assembly 700a includes L-shaped extension arm 730 which is mounted with respect to template body 702 through appropriate securement means (not pictured). For example, a locking screw may be introduced through aperture 738 to releasably lock extension arm 730 relative to template body 702. The L-shaped extension arm 730 includes a substantially planar extension region 732 and a downwardly extending region 734 with a plurality of positioning apertures 736 defined therein. Of note, downwardly extending region 734 defines an arcuate geometry, but the present disclosure is not limited to such geometry.

In use, the positioning apertures 708 associated with template body 702 and, in the case of template assembly 700a, positioning apertures 736 associated with downwardly extending region 734, allow the clinician to introduce a desired number of pins into the underlying anatomical structure (e.g., bone) to secure template assembly 700, 700a relative thereto. Thus, in exemplary implementations, a plurality of pins (not pictured) are introduced through mounting apertures 708 and/or mounting apertures 736 so as to achieve a desired level of security/stability.

Once secured to a desired anatomical site, the template assembly 700, 700a is generally used according to the present disclosure to guide a removal tool in creating a defect of a desired geometry, i.e., through interaction with the geometry of template member 704. The depth of the defect may be controlled in the manner described above with reference to previous embodiments.

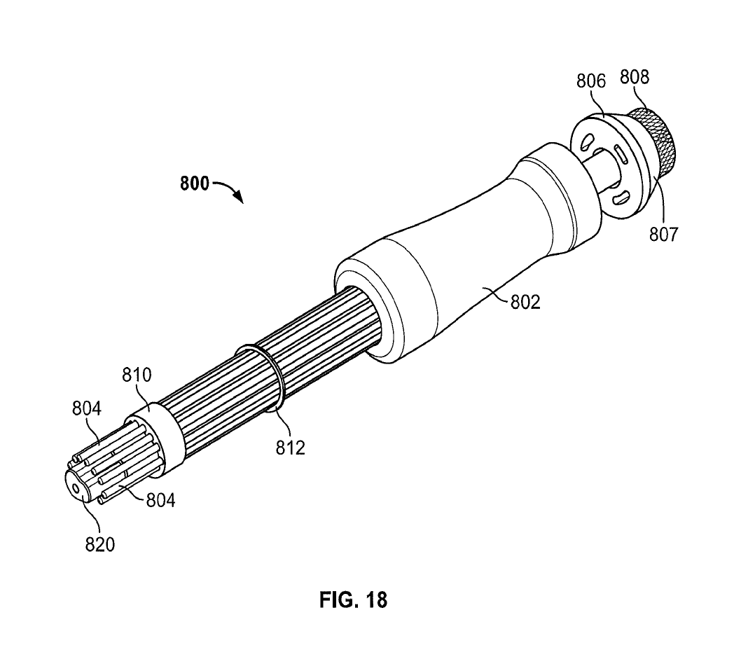

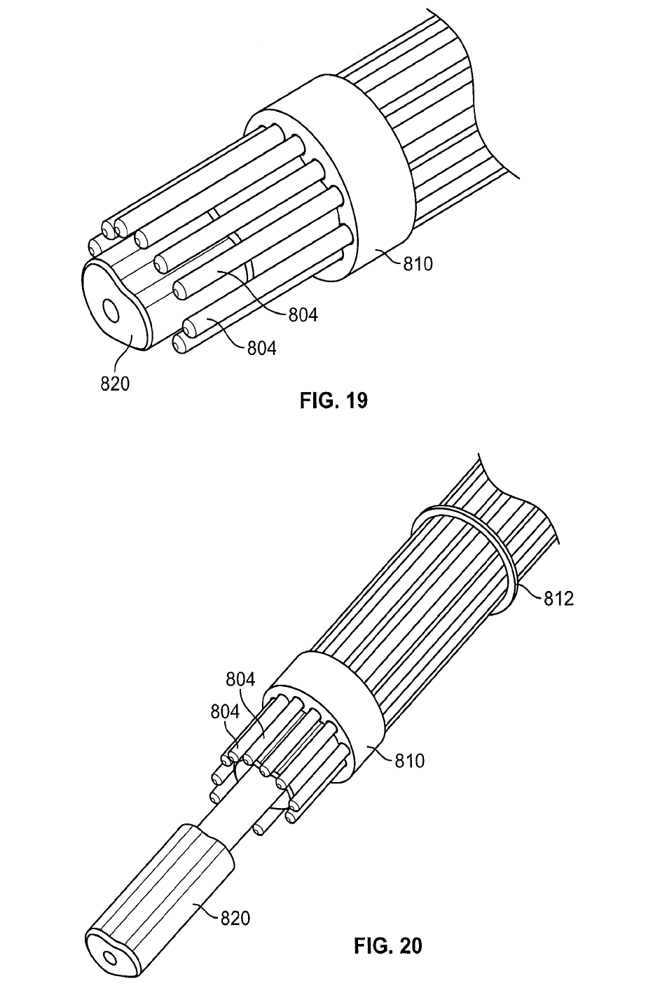

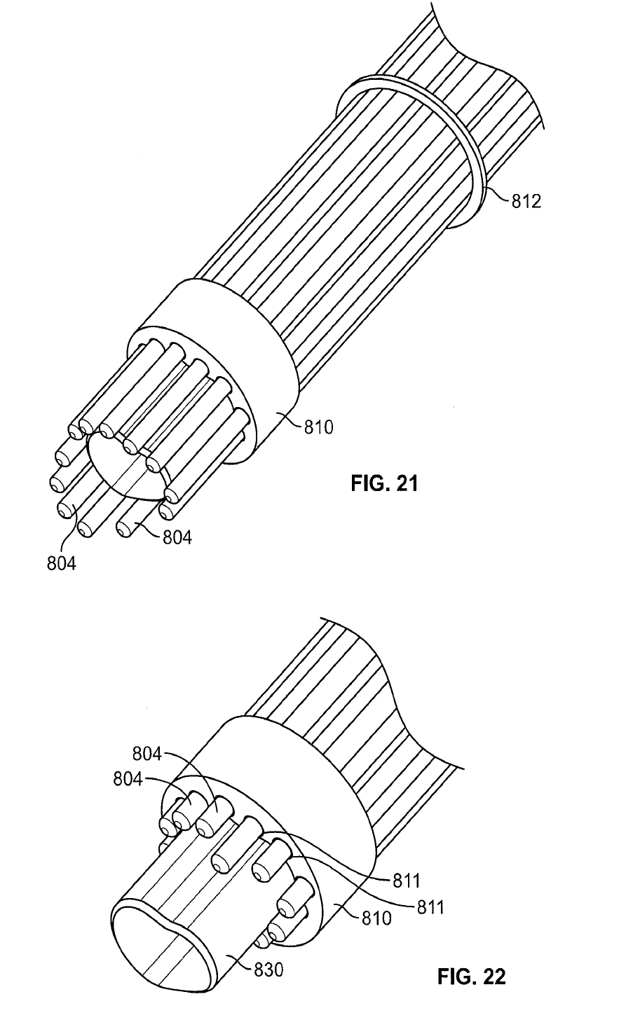

Turning to FIGS. 18-23B, an exemplary graft harvesting device 800 according to the present disclosure is depicted. Graft harvesting device 800 includes a handle member 802, a plurality of axially extending pins 804, a proximally positioned reset collar 806 and a proximally positioned anvil 807. Handle member 802 may be bulbous in geometry so as to facilitate manual interaction therewith. Pins 804 extend through handle member 802, e.g., through channels defined therein, and are radially deployed in a substantially circular geometry. Collar 810 also defines a series of radially spaced apertures 811 (best seen in FIG. 22) that serve to align/guide pins 804 at the distal end of graft harvesting device 800. An elastic ring 812 is deployed between handle member 802 and collar 810 to further stabilize pins 804 and to apply a further frictional force thereto.

Graft harvesting device 800 defines an interior channel that is adapted to receive one or more instruments and/or devices. Thus, with reference to FIGS. 18 and 19, a trial device 820 extends through the interior channel for purposes described herein below. Also, with reference to FIG. 22, a cutting member 830 extends through the interior channel of graft harvesting device 800.

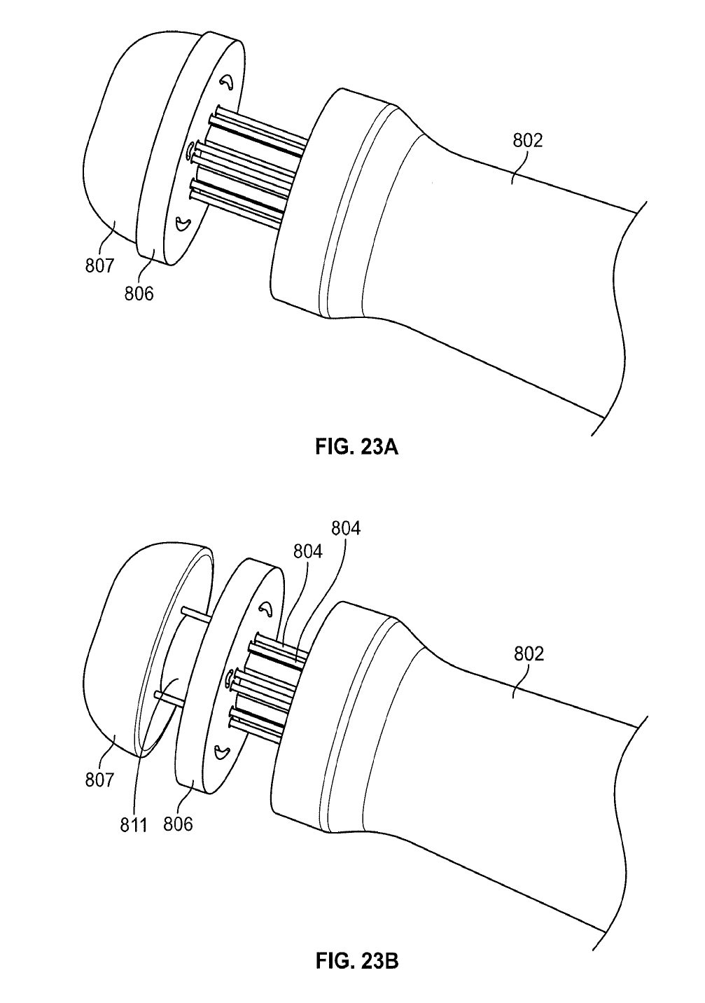

With reference to FIGS. 23A and 23B, the proximal end of exemplary graft harvesting device 800 is depicted. Of note, anvil 807 includes a distally extending cylindrical member 811 that defines an interior passage for receipt of ancillary devices/instruments. Cylindrical member 811 extends within the interior channel defined by graft harvesting device. Thus, for example, trial device 820 is removably received therethrough.

Of primary significance with respect to graft harvesting device 800 is the design/operation of pins 804. In particular, pins 804 are effective for capturing information concerning anatomical topography in the region of a defect and/or planned graft harvest. Pins 804 are adapted to slide axially relative to handle member 802 and, by positioning the distal ends of pins against an anatomical surface, it is possible to capture the topography thereof based on the relative proximal movement of the radially-deployed pins 804. Indeed, as seen in FIGS. 19-22, pins 804 reflect the topography of the anatomical region-of-interest. The relative position of pins 804 is generally preserved through frictional interaction/engagement with collar 810 and/or handle 802 (as well as elastic member 812), although positive locking mechanisms may be introduced to the graft harvesting device 800 if desired.

Thus, in an exemplary implementation of the present disclosure, graft harvesting device 800 is brought into proximity with a defect to be filled. Of note, trial device 820 may be advantageously part of an instrument set that features the same defect geometry. The instrument set generally includes a trial device (e.g., trial device 820), a template member (e.g., template member 704), and a cutting device (e.g., cutting member 830). The trial device 820 may be introduced to a defect defined using template assembly 700 (and template member 704) to confirm the geometry/orientation thereof. Thus, as shown in FIG. 19, the pins 804 assume relative positioning that reflects the topography adjacent the defect defined using template member 704, and the trial device 820 reflects the orientation of such defect relative to pins 804.

Thereafter, the trial device 820 may be removed from graft harvesting device 800 and cutting member 830 introduced therewithin. The pins 804 may be used to identify a harvest location that features a topography that corresponds to the topography surrounding the defect to receive the harvested graft. Cutting member 830 advantageously features the same geometry as template member 704 and trial device 820, thereby ensuring that the graft to be harvested will advantageously fit snugly within the previously-defined defect. As shown in FIG. 22, the pins are typically withdrawn in a proximal direction relative to collar 810 to facilitate the cutting/graft harvesting operation. Once the graft is harvested with by graft harvesting device 800, it may be delivered to the defect in the manner described above.

With reference to FIGS. 23A and 23B, the reset collar 806 may be used to reset the pins 804 after completion of a harvesting operation, thereby resetting the graft harvesting device 800 for reuse. Resetting of the pins 804 is generally accomplished by sliding the reset collar 806 distally relative to handle 802, as reflected in the distal movement as between FIG. 23A and FIG. 23B.

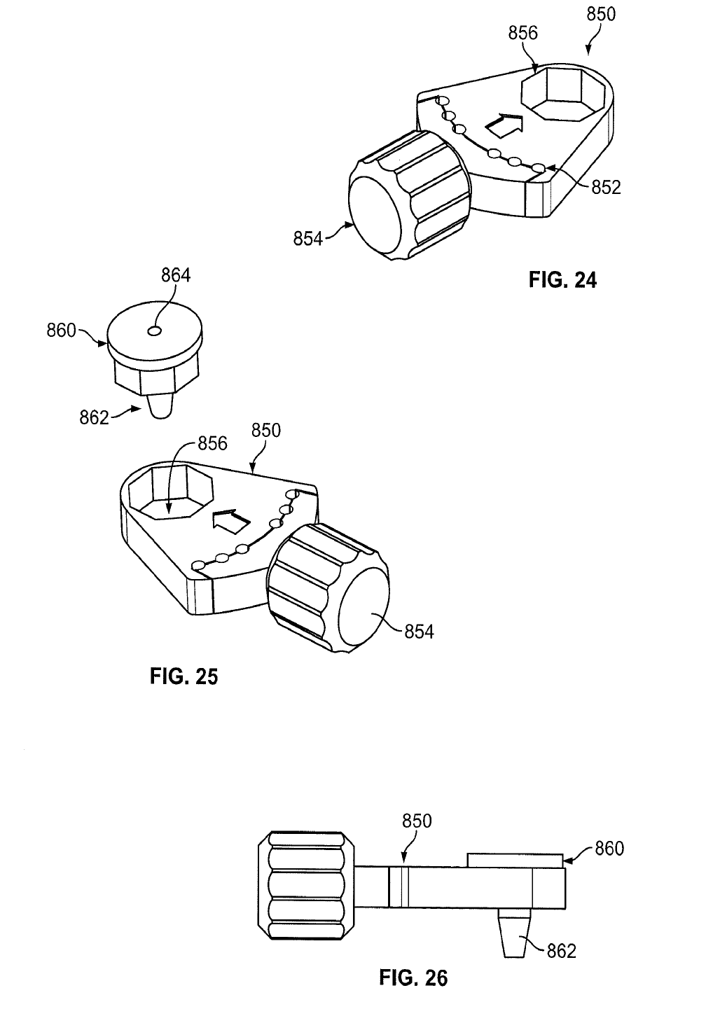

Turning to FIG. 24, a further exemplary fixture clamp 850 according to the present disclosure is depicted. Fixture clamp 850 includes a plurality of arcuately spaced holes 852 for receipt of K-wires (not pictured) so as to fix the fixture clamp 850 relative to an anatomical location. The number of K-wires utilized to position fixture clamp 850 relative to an anatomical location may vary from implementation-to-implementation, but generally only two K-wires are required for fixation purposes. Once the wires are positioned in the desired K-wire holes, clamping knob 854 is tightened down on the K-wires, thereby fixing the orientation/positioning of the fixture clamp 850 relative to the K-wires and, therefore, relative to the underlying anatomical structure. The body of the fixture clamp also defines a polygonal (e.g., hexagonal) opening 856 that is adapted to receive components associated with the disclosed system/methodology, as described in greater detail below.

Turning to FIGS. 25-26, the fixture clamp 850 is depicted with a pointer 860 positioned in the hexagonal opening 856 referenced above. The pointer 860 includes a downwardly projecting extension 862 that is advantageously employed to locate a defect and to position the overall system/apparatus relative thereto. Of note, the pointer 860 includes a central aperture 864 is adapted to receive a K-wire therethrough. Thus, in an exemplary implementation of the present disclosure, the following procedural steps are undertaken:
a K-wire is positioned in a defect of interest;
the pointer 860 is positioned in the hexagonal opening 856 of the fixture clamp 850;
the K-wire is slid through the pointer 860, thereby aligning the center of the hexagonal opening 856 with the location of the K-wire (and the associated defect);
the fixture clamp 850 is slid onto the K-wire and then at least two additional K-wires are introduced into the arcuately spaced holes 852 formed in the fixture clamp 850;
the clamping knob 854 is tightened, thereby fixing the fixture clamp 850 relative to the at least two additional K-wires; and
the defect-locating K-wire and the pointer 860 may now be removed because the fixture clamp 850 is fixedly located relative to the defect with the hexagonal opening 856 positioned directly thereover.

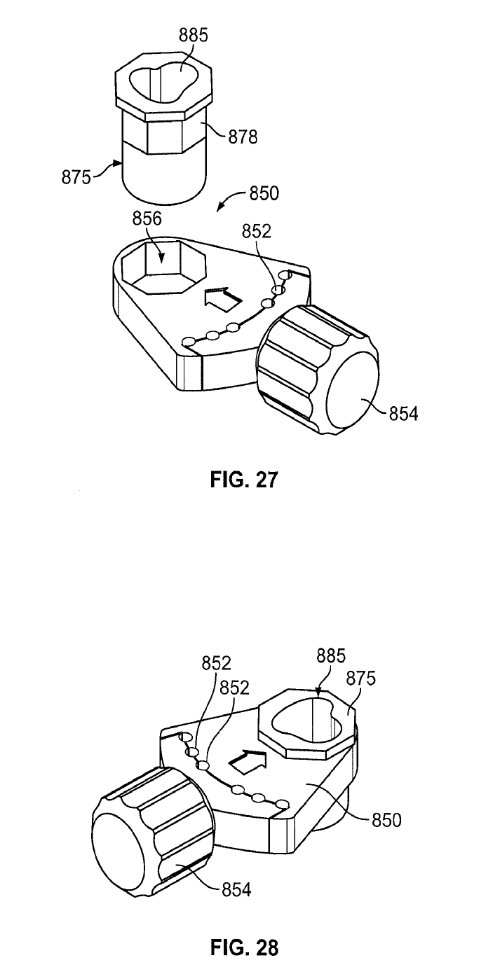

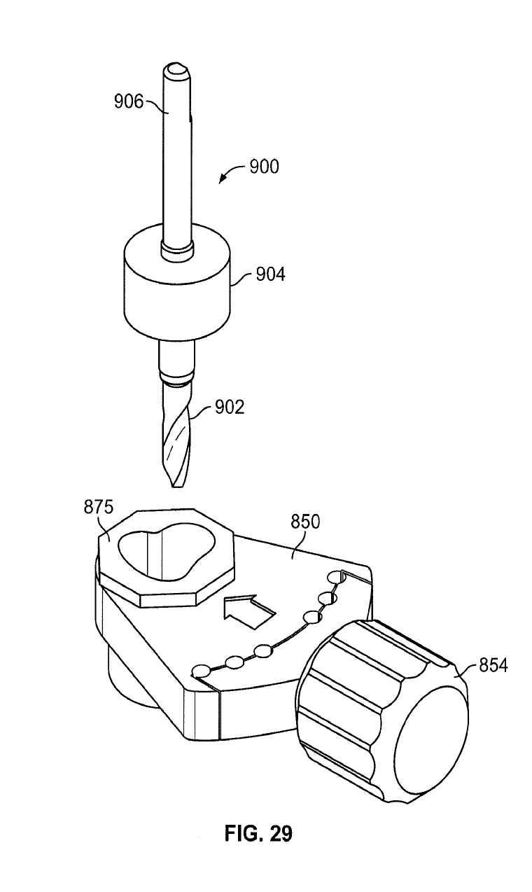

With reference to FIGS. 27-28, a defect template member 875 is next selected by the clinician and positioned in the hexagonal opening 856 of the fixture clamp 850. Of note, the template member 875 features a circumferential surface 878 that matches the hexagonal opening 856 formed in the fixture clamp 850, thereby keying the template member 875 relative to the fixture clamp 850. Template member 875 defines a template geometry 885 that corresponds to a desired defect geometry.

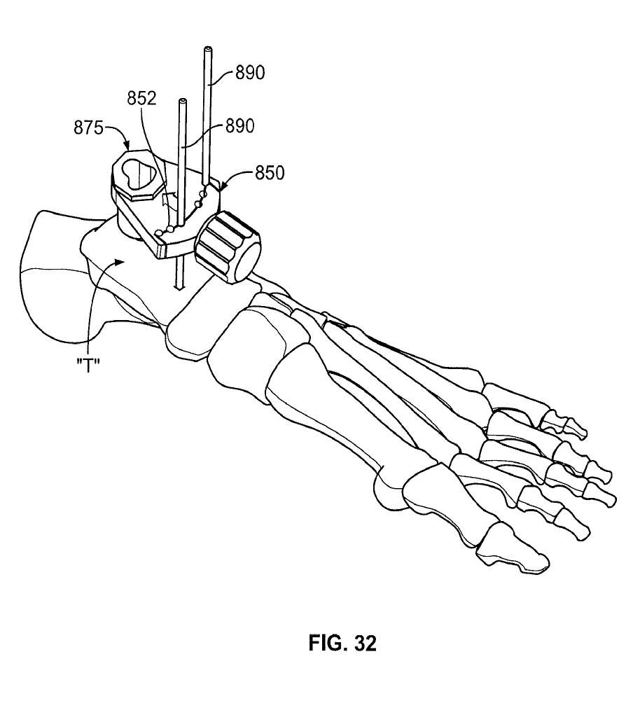

With reference to FIG. 32, the noted fixture clamp 850 and template member 875 are shown mounted with respect to a talus “T” using a pair of K-wires 890 that pass through spaced apertures 852 defined in fixture clamp 850.

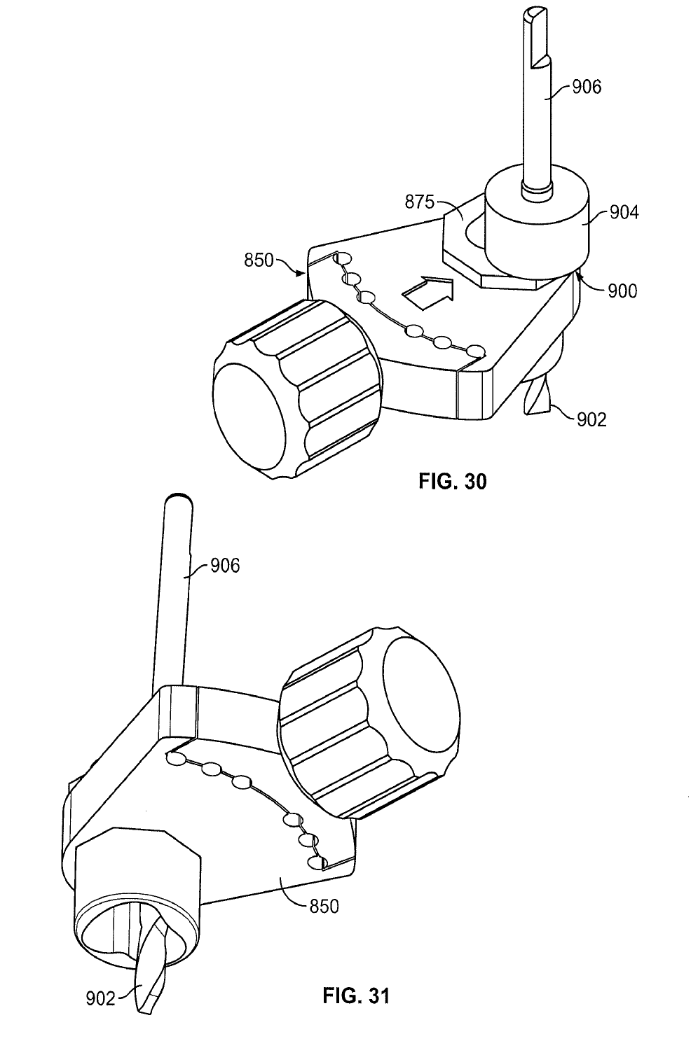

Turning to FIGS. 29-31, a cutter 900 that includes a cutting bit 902 may be advantageously introduced through the defect template member 875 to cut a defect region of a desired geometric shape into the underlying structure. The cutting depth is controlled by a guide bushing 904 or like structure that is mounted with respect to the cutting bit 902 and controls the degree to which the cutting bit 902 can penetrate the underlying structure. Cutting bit 902 communicates with a drive shaft 906 that is adapted to cooperate with a drive mechanism (not pictured), as is known in the art. After the cutter 900 is employed to create a defect region that corresponds to template geometry 885 (of a desired depth based on interaction between guide bushing 904 and fixture clamp 850, the fixture clamp 850 may be removed from the clinical field.

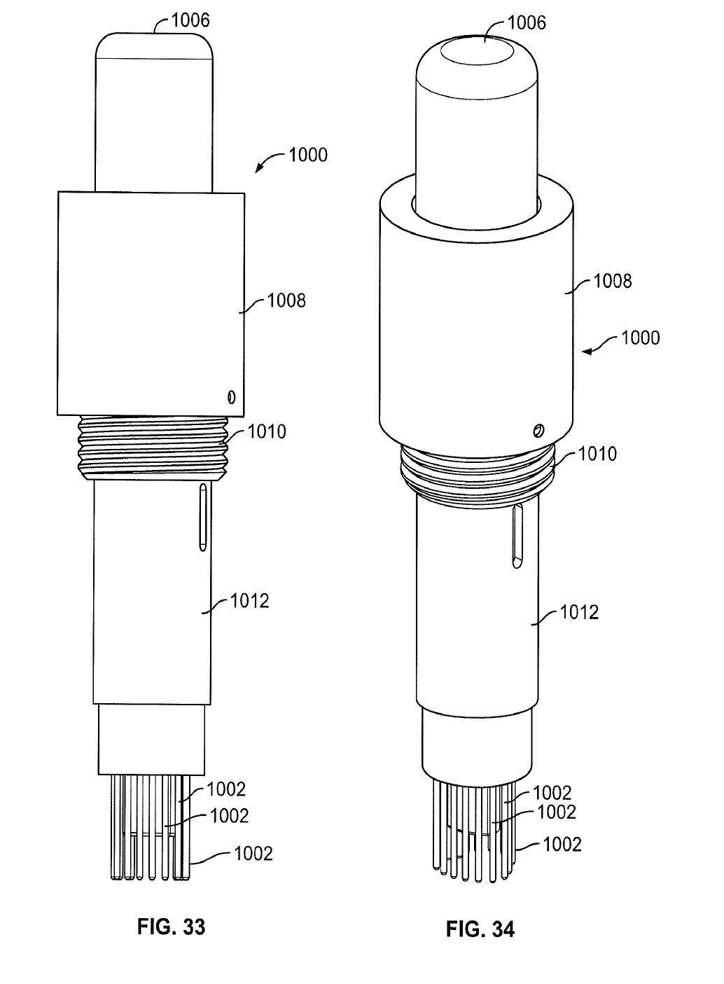

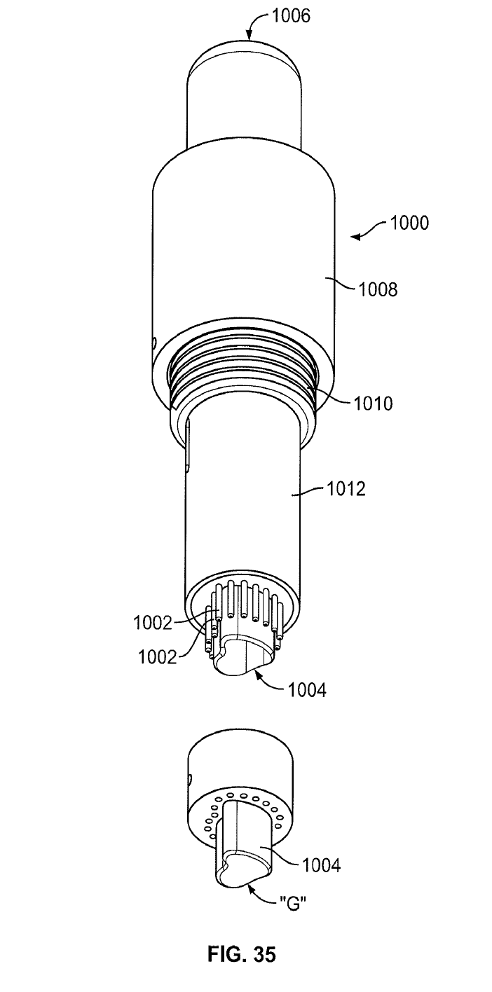

With reference to FIGS. 33-35, an exemplary pin guide and punch assembly 1000 is disclosed for use in capturing the topography of the region surrounding a defect and then acquiring an implant for introduction to the defect-of-interest. The pins 1002 associated with assembly 1000 generally operate in the manner described above, e.g., with reference to FIGS. 4A, 4B and 4D, and such operation will not be described again herein with reference to FIGS. 33-35. Once the topography is captured by the pins 1002, the pin guide and punch assembly 1000 can be positioned with respect to graft material so as to identify a region with comparable topographic characteristics. This effort may be guided using mapping techniques and systems of the type set forth in commonly assigned PCT application entitled “Systems, Devices and Methods for Cartilage and Bone Grafting” (WO 2009/154691 A9; corrected version).

A punch 1004 is associated with the pin guide and punch assembly 1000. Punch 1004 defines a geometry corresponding to a corresponding template geometry, e.g., template geometry 856. At the proximal end of punch 1004 is an impact face 1006 that can be impacted to drive the punch 1004 into desired bone/cartilage. The pin guide and punch assembly 1000 further includes an axially translatable sleeve 1008 that is adapted to advance/retract relative to the longitudinal axis of assembly 1000. As shown in FIGS. 33-35, sleeve 1008 includes internal threads or an inwardly directed pin (not pictured) adapted for axial translation relative to outwardly directed threads 1010 formed on body 1012. Axial translation of sleeve 1008 is adapted to deliver a downward force within punch 1004 so as to advance graft plug “G” from the distal end thereof.

Once a graft plug “G” is acquired using the punch 1004, the clinician can introduce such plug “G” to the enlarged defect (and tamp it into place). The plug is positioned with the pins 1002 and advantageously defines a geometry that substantially matches the geometry of the defect template 856. Thus, the plug “G” fits within the enlarged defect while topography information is captured by the pins 1002.

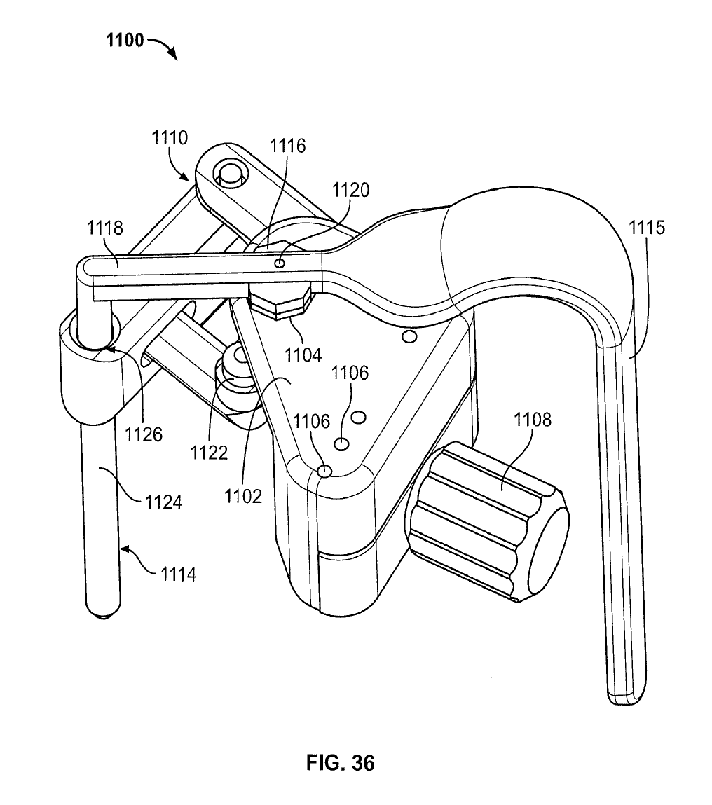

Turning to FIGS. 36-40, an alternative exemplary implementation of the present disclosure is provided by system 1100 that utilizes elements of the previously described embodiment, i.e., the fixture clamp. With initial reference to FIG. 36, system 1100 includes a fixture clamp 1102 that defines a hexagonal opening 1104 and a plurality of holes 1106 for receipt of K-wires (not pictured). A clamping knob 1108 is provided for tightening relative to such K-wires when the fixture clamp 1102 is in a desired orientation. However, unlike previous embodiments, the disclosed fixture clamp 1102 cooperates with a four-bar linkage mechanism 1110, whereby the fixture clamp 1102 is adapted to be positioned/oriented at a fixed distance—defined by a pointer subassembly 1114—relative to the defect. The pointer subassembly includes a handle 1115, an intermediate fitting 1116 for receipt within the noted hexagonal opening 1104 of the fixture clamp 1102, but also includes an extension arm 1118 that extends therebeyond. The intermediate fitting 1116 is adapted to engage an upstanding pin 1120 that extends upward through the hexagonal opening 1104 from the underlying linkage arm 1122 (associated with the four-bar linkage mechanism 1110). Thus, the intermediate fitting 1116 temporarily locks the four-bar linkage mechanism 1110 in a fixed position. At the distal end of the extension arm 1118 associated with the pointer subassembly 1114, a downwardly directed pointer 1124 is adapted to pass through a guide channel 1126 formed at the end of the four-bar linkage mechanism 1110.

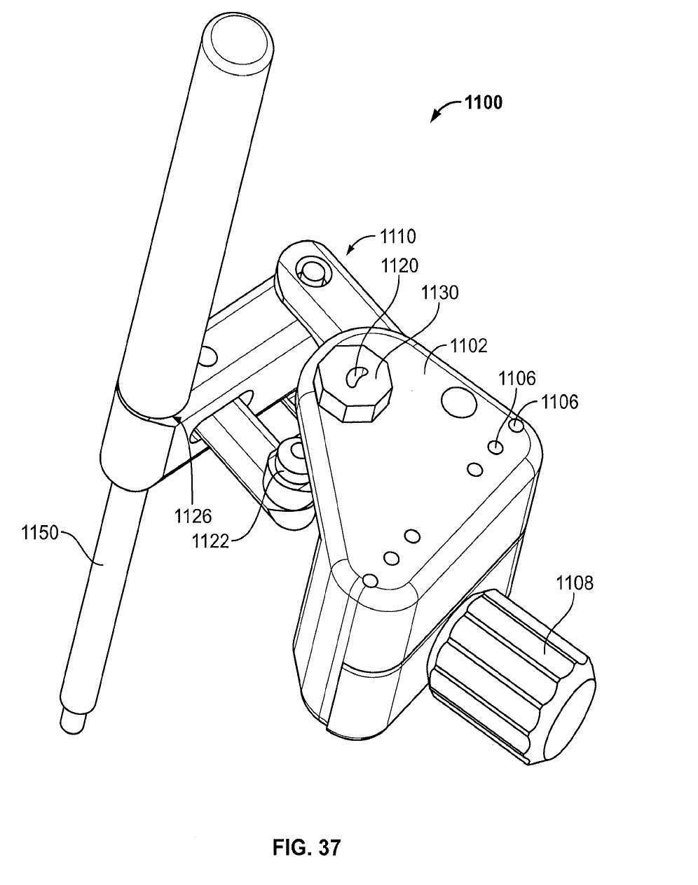

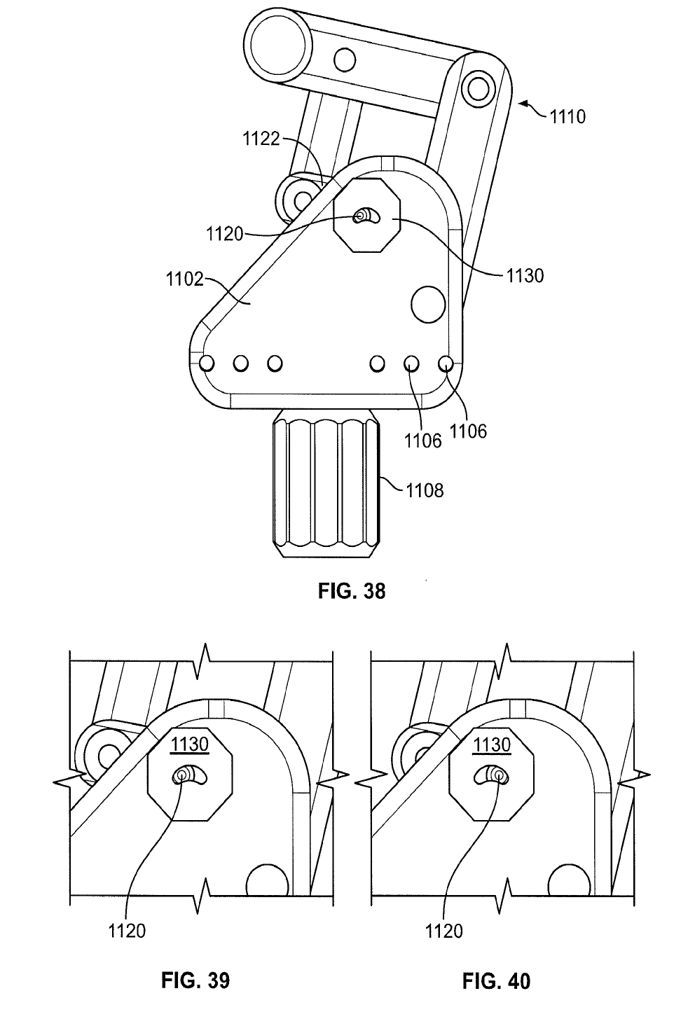

In use, the pointer 1124 is positioned within the guide channel 1126 above a desired defect and the clamping knob 1108 is tightened down on the K-wires (not pictured), thereby fixing the fixture clamp 1102 relative to the defect. The pointer subassembly 1114 can then be removed and, as shown in FIGS. 37-39, a desired defect template 1130 inserted into the hexagonal opening 1104. Of note and with reference to FIGS. 38-40, the pin 1120 within the defect template 1130 extends upward from the underlying linkage 1122 associated with the noted four-bar linkage mechanism 1110. Thus, movement of the pin 1120 within the defect template 1130 necessarily translates to corresponding (but amplified) motion of the four-bar linkage mechanism 1110, with the motion being replicated (but amplified) at the center point of the guide channel 1126.

As shown in FIG. 37, with the defect template 1130 in place, a cutter 1150 is positioned in the guide channel 1126 and by moving the pin 1120 within the defect template 1130, the desired cutting geometry will be achieved below the guide channel 1126. The ratio of the template opening formed in defect template 1130 relative to the corresponding motion at the guide channel 1126 is predetermined based on the design of the disclosed assembly 1100. In an exemplary embodiment, the ratio of movement at the guide channel 1126 relative to movement of pin 1120 within the defect template 1130 is about 3:1, although the present disclosure is not limited by or to such implementation. Of note, the alternative embodiment of FIGS. 36-40 offers enhanced visualization for system users since the operative activity is spaced from the fixture clamp 1102 and associated components.

Although the present disclosure has been described with reference to exemplary embodiments and implementations thereof, the disclosed embodiments/implementations are illustrative in nature. Thus, the present disclosure encompasses and extends to variations, modifications and enhancements that would be readily apparent to persons skilled in the art in view of the present disclosure and therefore fall within both the scope and spirit of the present disclosure.

1. A system for use in defect repair, comprising:
an instrument for forming a defect region of a predetermined geometry, the instrument including: (i) means for establishing referential orientation relative to an anatomical location; (ii) means for controlling geometry of material removal at the anatomical location; and (iii) means for capturing information concerning surface contour of the anatomical location in proximity to the defect region.

2. The system of claim 1, further comprising means for removing material to define the defect region.

3. The system of claim 1, wherein the means for establishing referential orientation includes a cannula that is adapted to be positioned relative to a target location.

4. The system of claim 3, wherein the cannula includes a mounting mechanism for use in rigidly mounting the cannula relative to a fixed structure.

5. The system of claim 1, wherein the means for controlling geometry of material removal includes a template member that defines an opening corresponding to a desired material removal geometry.

6. The system of claim 1, further comprising means to control depth of material removal from the anatomical location.

7. The system of claim 6, wherein the means for controlling depth of material removal includes a bushing member.

8. The system of claim 1, wherein the means for capturing surface contour information includes a plurality of axially movable pins that are adapted to move independently of one another.

9. The system of claim 1, further comprising means for harvesting a plug of material from an anatomical site that is adapted to cooperate with the instrument.

10. The system of claim 9, wherein the harvesting means is adapted to cooperate with the means for capturing surface contour information so as to harvest a plug of material that includes a surface geometry that substantially corresponds to the surface contour of the anatomical location.

11. The system of claim 10, wherein the harvesting means and the means for capturing surface contour information cooperate through a keying mechanism.

12. (canceled)

13. The system of claim 1, further comprising a trial member and a cutter, and wherein the means for controlling geometry of material removal, the trial member and the cutter define a set with matching defect-related geometric properties.

14. A method for defect repair, comprising:
a) establishing referential orientation of an instrument relative to an anatomical location;
b) forming a defect region of predefined geometry in the anatomical location;
c) capturing information concerning surface contour of the anatomical location in the vicinity of the defect region; and
d) using the captured information to identify a donor location with a complementary surface contour as a harvest region with a complementary surface contour for excision of a plug to fill the defect region.

15. The method of claim 14, wherein the defect region is formed with a predefined depth.

16. The method of claim 14, further comprising obtaining a plug from the harvest region and introducing the plug into the defect region.

17. The method of claim 16, wherein (i) the defect region is formed using a template having a predefined opening geometry, (ii) the plug is obtained using a cutter having a cutting geometry; and (iii) the predefined opening geometry of the template and the cutting geometry of the cutter correspond to each other.

18. The method of claim 14, wherein the defect region is formed at substantially a right angle relative to the axis of the instrument used to form such defect region.

19. An instrument for use in defect repair, comprising:
an elongated shaft;
a cutting member mounted with respect to the elongated shaft and operative to form a defect region of predetermined geometry;
wherein the cutting member is operative at an angle of about 90 degrees relative to the elongated shaft.

20. The instrument of claim 19, further comprising a template mounted with respect to the elongated shaft, and wherein the cutting member is operative to form a defect region that substantially corresponds to the template.

21. The instrument of claim 19, wherein the cutting member is adapted to move between a non-deployed and a deployed relative to the elongated shaft.

22. The instrument of claim 19, further comprising a drive mechanism that drives operation of the cutting member.

23. The instrument of claim 22, wherein the drive mechanism is selected from the group consisting of (i) a drive shaft that includes at least one bevel gear;
(ii) a drive system that includes a conduit for, high pressure fluid flow; (iii) a drive system that includes a belt and pulley mechanism.

24. The system of claim 1, further comprising
a four-bar linkage mechanism for translating the controlled geometry to a desired anatomical location.

25. The system of claim 24, further comprising a cutting device that cooperates with the four-bar linkage mechanism to form a defect region corresponding to the controlled geometry at a desired anatomical location.
